(19) United States
(12) Patent Application Publication
(10) Pub. No.: US 2017/0226478 A1
(43) Pub. Date: Aug. 10, 2017

(54) Neuromuscular Junction: NMJ-ON-CHIP

(71) Applicants: EMULATE, INC., Boston, MA (US); CEDARS-SINAI MEDICAL CENTER, Los Angeles, CA (US)

(72) Inventors: Jordan Kerns, Reading, MA (US); Norman Wen, West Roxbury, MA (US); Geraldine Hamilton, Boston, MA (US); Christopher Hinojosa, Cambridge, MA (US); Jacob Fraser, Somerville, MA (US); Catherine Karalis, Brookline, MA (US); Janna Nawroth, Boston, MA (US); Dhruv Sareen, Porter Ranch, CA (US); Anjoscha Kaus, Los Angeles, CA (US); Berhan Mandefro, Sherman Oaks, CA (US); Hyoung Shin Park, Newton, MA (US); Ville Kujala, Medford, MA (US)

(21) Appl. No.: 15/458,185

(22) Filed: Mar. 14, 2017

Related U.S. Application Data
(63) Continuation-in-part of application No. 15/352,289, filed on Nov. 15, 2016.
Application No. 15/352,289 is a continuation of application No. PCT/US2016/057724, filed on Oct. 19, 2016.
(60) Provisional application No. 62/380,780, filed on Aug. 29, 2016.
(60) Provisional application No. 62/332,727, filed on May 6, 2016.
(60) Provisional application No. 62/277,723, filed on Jan. 12, 2016.
(60) Provisional application No. 62/243,642, filed on Oct. 19, 2015.

(51) Int. Cl. C12N 5/0793; B01L 3/00; C12N 5/077

(52) U.S. Cl. CPC C12N 5/0619; C12N 5/0658; B01L 3/502761; C12N 2506/45; C12N 2529/00; C12N 2539/00; C12N 2502/00; B01L 2200/0647; B01L 2200/10; B01L 2300/06

(57) ABSTRACT

The invention relates to culturing motor neuron cells together with skeletal muscle cells in a fluidic device under conditions whereby the interaction of these cells mimic the structure and function of the neuromuscular junction (NMJ) providing a NMJ-on-chip. Good viability, formation of myo-fibers and function of skeletal muscle cells on fluidic chips allow for measurements of muscle cell contractions. Embodiments of motor neurons co-cultures with contractile myo-fibers are contemplated for use with modeling diseases affecting NMJ's, e.g. Amyotrophic lateral sclerosis (ALS).

### FIELD OF THE INVENTION

The invention relates to culturing motor neuron cells together with skeletal muscle cells in a microfluidic device under conditions whereby the interaction of these cells mimic the structure and function of the neuromuscular junction (NMJ) providing a NMJ-on-chip. Good viability, formation of myo-fibers and function of skeletal muscle cells on fluidic chips allow for measurements of muscle cell contractions. Embodiments of motor neurons co-cultures with contractile myo-fibers are contemplated for use with modeling diseases affecting NMJ's, e.g. Amyotrophic lateral sclerosis (ALS).

### BACKGROUND OF THE INVENTION

The neuromuscular junction (NMJ) is of major clinical relevance. First, dysfunction of the NMJ leads to degeneration of motor neuron-skeletal muscle unit. Secondly, drugs that are supposed to treat neurological disorders often fail to restore the end plate potential to activate the muscle fibers.

Amyotrophic lateral sclerosis (ALS) is most common neurodegenerative disease affecting 2.5 in 100,000 per year but the cause of the disease is unknown.

Because of its importance in disease and medical treatment, it would be highly advantageous to have a predictive model of the NMJ that recapitulates aspects of the motoneuronal-muscle cell microenvironment in a controlled way.

### SUMMARY OF THE INVENTION

The invention relates to culturing motor neuron cells together with skeletal muscle cells in a fluidic device under conditions whereby the interaction of these cells mimic the structure and function of the neuromuscular junction (NMJ). Good viability, formation of myo-fibers and function of skeletal muscle cells on fluidic chips allow for measurements of muscle cell contractions. Embodiments of motor neurons co-cultures with contractile myo-fibers are contemplated for use with modeling diseases affecting NMJ's, e.g. Amyotrophic lateral sclerosis (ALS).

In one embodiment, the present invention contemplates a method of culturing cells, comprising: a) providing a microfluidic device comprising a membrane, said membrane comprising a top surface and a bottom surface; b) seeding induced motor neuron cells on said top surface and skeletal muscle cells on said bottom surface so as to create seeded cells; c) exposing said seeded cells to a flow of culture media for a period of time; and d) culturing said seeded cells under conditions such that a neuromuscular junction forms within said microfluidic device. The formation of the neuromuscular junction can be detected in a number of ways. It is not intended that the present invention be limited to how the neuromuscular junction is detected or measured. In one embodiment, the NMJ detected by measurement and/or detection of the binding of α-bungarotoxin (BTX), Tubulin beta-3 chain (Tubb3) and/or muscle myosin heavy chain (MHC), and in a preferred embodiment, where co-localization of these markers is detected. In a preferred embodiment, a color label (e.g. fluorescent label) is used for each marker with combined multi-channel reading as a measurement of co-localization. However, the present invention contemplates additional approaches including but not limited to functional measurement/detection of the NMJ. Such functional embodiments include measuring and/or detecting the formation of the NMJ as demonstrated by measuring and/or detecting nerve action potential, neurotransmitter release, muscle cell membrane activation potential and/or myofiber contraction. In one embodiment, these events occur in sequence and are synchronized (e.g. with synchronization comparable to an in vivo neuromuscular junction response as understood to one of ordinary skill). In one embodiment, said skeletal muscle cells are induced to differentiate. In one embodiment, said skeletal muscle cells form contractile tissue. In one embodiment, said skeletal muscle cells form polynucleated myo-fibers. In one embodiment, said seeded cells are cultured for more than ten days. In one embodiment, said induced motor neuron cells are derived from induced pluripotent stem cells from a human. In one embodiment, said human is diagnosed with a CNS disorder. In one embodiment, the present invention contemplates that the method further comprises the step of e) assessing the health and/or integrity of the neuromuscular junction. This can be done a number of ways. For example, this can be done by measurement and/or detection of the binding of α-bungarotoxin (BTX), Tubulin beta-3 chain (Tubb3) and/or muscle myosin heavy chain (MHC), and in a preferred embodiment, where co-localization of these markers is detected. This can also be done by measuring and/or detecting nerve action potential, neurotransmitter release, muscle cell membrane activation potential and/or myofiber contraction. The present invention also contemplates and embodiment where the method further comprises the step of e) electrically stimulating said motor neurons and/or said skeletal muscle cells.

It is not intended that the present invention be limited to situations where both neurons and skeletal muscle cells are seeded together. In one embodiment, the present invention contemplates a method of culturing cells, comprising: a) providing a microfluidic device comprising a channel; b) seeding skeletal muscle cells into said channel; c) inducing said skeletal muscle cells to differentiate; and d) detecting myo-fiber formation. Motor neurons can be (optionally) added before or after the muscle cells (or not at all). In one embodiment, said detecting of myo-fiber formation comprises detecting myo-fiber contractions. In one embodiment, said seeded cells are exposed to a flow of culture media for a period of time. In a preferred embodiment, the cells are seeded onto covalently attached ECM protein(s).

The present invention also contemplates seeding on both patterned surfaces and/or gels. In one embodiment, the present invention contemplates a method of culturing cells, comprising: a) providing a microfluidic device comprising a patterned surface and a gel, b) seeding induced motor neuron cells on said patterned surface and skeletal muscle cells on said gel. In one embodiment, the present invention contemplates that the method further comprises c) detecting myo-fiber formation by said skeletal muscle cells. In one embodiment, said detecting of myo-fiber formation comprises detecting myo-fiber contractions. In one embodiment, said skeletal muscle cells and/or said motor neurons are exposed to a flow of culture media for a period of time.

The present invention also contemplates microfluidic devices with cells. In one embodiment, the present invention contemplates a microfluidic device comprising a) a membrane, said membrane comprising a top surface and a bottom surface; and b) induced motor neuron cells on said top surface and skeletal muscle cells on said bottom surface. In one embodiment, said induced motor neuron cells are derived from induced pluripotent stem cells from a human. In one embodiment, said human is diagnosed with a CNS disorder. In one embodiment, said CNS disorder is ALS. In one embodiment, said membrane comprises covalently attached ECM protein(s).

The present invention also contemplates systems comprising microfluidic devices with cells under flow conditions. In one embodiment, the present invention contemplates a system comprising a microfluidic device, said microfluidic device comprising a) a membrane, said membrane comprising a top surface and a bottom surface; and b) induced motor neuron cells on said top surface and skeletal muscle cells on said bottom surface, wherein either one of said cell types or both are exposed to culture media at a flow rate. In one embodiment, said induced motor neuron cells are derived from induced pluripotent stem cells from a human. In one embodiment, said human is diagnosed with a CNS disorder. In one embodiment, said CNS disorder is ALS. In one embodiment, said membrane comprises covalently attached ECM protein(s). In one embodiment, the membrane is in a channel, said channel is in fluidic communication with a reservoir comprising culture media.

### DEFINITIONS

Some abbreviations are used herein.

For example, “MN” refers to motor neurons. The letter “i” indicates “induced.” Thus, “iMN” indicates induced motor neurons, i.e. motor neurons that were induced or generated from other cells, e.g. stem cells. “diMN” indicates direct induced motor neurons. “iMNP” indicates induced motor neuron progenitor cells, which are not fully differentiated into mature neurons.

The term “microfluidic” as used herein relates to components where moving fluid is constrained in or directed through one or more channels wherein one or more dimensions are 10 mm or smaller (microscale). Microfluidic channels may be larger than microscale in one or more directions, though the channel(s) may be on the microscale in at least one direction. In some instances the geometry of a microfluidic channel may be configured to control the fluid flow rate through the channel. Microfluidic channels can be formed of various geometries to facilitate a wide range of flow rates through the channels. However, it is important to note that while the present disclosure makes frequent reference to “microfluidic” devices, much of what is taught applies similarly or equally to larger fluidic devices. Larger devices may be especially relevant if the “NMJ-on-chip” is intended for therapeutic application. Examples of applications that may make advantage of larger fluidic devices include the use of the device for the generation of highly differentiated cells (e.g. the device can used to drive cell differentiation and/or maturation, whereupon the cells are extracted for downstream use, which may include implantation, use in an extracorporeal device, or research use), or use of the device for implantation or extracorporeal use, for example, as an artificial NMJ. Unlike conventional static cultures, the present invention contemplates microfluidic devices where the cells are exposed to a constant flow of media providing nutrients and removing waste.

As used herein, the phrases “connected to,” “coupled to,” and “in communication with” refer to any form of interaction between two or more entities, including mechanical, electrical, magnetic, electromagnetic, fluidic, and thermal interaction. For example, in one embodiment, first and second channels in a microfluidic device are in fluidic communication with a fluid reservoir. Two components may be coupled to each other even though they are not in direct contact with each other. For example, two components may be coupled to each other through an intermediate component (e.g. tubing or other conduit).

### BRIEF DESCRIPTION OF THE DRAWINGS

FIG. 1A-1C: shows schematics of neuromuscular junctions (NMJs) as interfaces between spinal motor neurons and skeletal muscle cells.

FIG. 1A: shows a schematic illustration of the exterior of neuromuscular junctions where the yellow axon of a motor nerve at the motor junction has non-myelinated terminal nerve branches fanning neuromuscular junctions (one example of an NMJ is outlined by a square). The neuronal terminal nerve branches have synaptic end bulbs or boutons (see FIG. 1B) located opposite of a muscular fiber end plate (see FIG. 1B). FIG. 1A also shows a schematic of an interior view of a muscle fiber composed of numerous myo-fibers interspersed with mitochondria (blue), sarcoplasmic reticulum (yellow tubes) within the sarcoplasm of a muscle fiber cell (myocyte).

FIG. 1B: shows a cut-out schematic illustration of the interface between spinal motor neurons and skeletal muscle cells, e.g., a NMJ, for demonstrating the steps of normal motor neuronal activation of muscle fibers. Step 1) An action potential of a myelinated axon reaches the non-myelinated axon terminal branch. Step 2) Voltage-dependent calcium gates open allow Ca++ to enter the end bulb which in Step 3) induces the movement of neurotransmitter containing vesicles to merge with the cell membrane at the end of the synaptic bulb opposite muscle cell acetylcholine (ACh) receptors located in the motor end plates. Neurotransmitter vesicles containing acetylcholine (ACh) are emptied (by exocytosis) into the synaptic cleft. Step 4) Freed ACh from the vesicles then diffuses across the cleft to bind to postsynaptic receptors on the sarcolemma of the muscle fiber in the motor end plate area. Step 5) This ACh binding causes ion channel pumps to open which allows sodium ions to flow across the membrane into the muscle cell while fewer K+ ions are transported out of the cell i.e. (3) Na+ ions enter the cell cytoplasm while (2) K+ ions are transported out, thus triggering a post synaptic action potential (end plate potential) in the NMJ, i.e. the end plate of the muscle sarcolemma. Step 6) the postsynaptic action potential (AP) generated at the end plate, Step 7) AP wave, i.e., sarcolemma membrane depolarization, travels across the muscle cell membrane.

FIG. 1C: shows a schematic illustration of a muscle cell (myocyte) depicting how the postsynaptic action potential (AP), triggered by the NMJ, in the sarcolemma of the motor end plate, in Step 6) travels to nearby areas of the T-tubules (i.e. a wave of ion pump activation that travels along the membrane whereby (3) Na+ ions enter the cell cytoplasm while (2) K+ ions are transported out of the cell cytoplasm. Further in Step 7) When the AP reaches areas of the T-tubule portion of the sarcolemma, destabilizing this area of the membrane, the AP in the sarcolemma of the T-tubule area causes the T-tubule to induce the release of Ca++ from the sarcoplasmic reticulum. Step 8) The destabilized sarcolemma then triggers a wave of Ca++ release across the sarcoplasmic reticulum membrane inside of the myocyte. Step 9) The rise in intracellular Ca++ activates contraction of myofibrils, i.e. myosin-actin interactions.

FIG. 2A-2D: shows 2-Dimensional (2D) motor neurons (MN) and muscle cell co-cultures grown in static plates, on day 37 of culture.

FIG. 2A: shows a micrograph of healthy human muscle skeletal cells (hSkMCs);

FIG. 2B: shows a higher magnification of cells in FIG. 2A, where the green arrow points to one exemplary multi-nucleated myotube;

FIG. 2C: shows a micrograph of a co-culture of direct induced motor neurons (diMNs) on top of hSkMCs where white arrows point to rounded cell bodies, a green arrow points to an exemplary myotube and a red arrow points to an exemplary neuron on top of said myotube; and

FIG. 2D: shows a higher magnification of cells in FIG. 2C where the red arrow points to neuronal branches on top of a myotube identified by a green arrow. White boxes outline the areas shown in higher magnification.

FIG. 3A-3B shows exemplary phase contrast images for embodiments of neuronal growth.

FIG. 3A: shows iMNs seeded on a plain (un-patterned) surface; and

FIG. 3B: shows a duplicate sample of cells (as in FIG. 3A) that were seeded on a nanopatterned surface, resulting in directed neurite growth.

FIG. 4A-4C: Shows one embodiment of a human skeletal muscle cell culture hSkMC-In-Chip: Extracellular Matrix for fluidic hSkMCs-In-Chip. In one embodiment, the chip is a Quad chip.

FIG. 4A: shows a picture of a single channel (Quad) Chip with pipette tips used to block channels for coating the inside surfaces with an ECM layer then seeded with human skeletal muscle cells (hSKMCs).

FIG. 4B: shows a schematic illustration of a cross-sectional view of the quad channel with ECM as Laminine (purple and blue stars) with hSkMCs as blue spotted yellow blocks.

FIG. 4C: shows a schematic illustration of a cross-sectional view of the quad channel with ECM as Laminine (purple and blue stars) with hSkMCs as blue spotted yellow blocks and a representative cross linking of ECM as yellow stars, e.g. with Sulfo-SANPAH.

FIG. 5A-5D: shows one embodiment of a human muscle cell culture in-chip: Set Up and Time Course for producing multinucleated myofibers that are not contracting.

FIG. 5A: Single channels of Quad Chips were seeded with human skeletal muscle cells (hSKMCs). Group 1 and Group 2: 5×10⁶/ml cells; Group 3 and Group 4: 1.6×10⁶/ml cells. Groups 1 and 3 do not have cross (X)-linked ECM while Groups 2 and 4 have exemplary Sulpho SANPA X-linked ECM.

FIG. 5B: shows a schematic experimental timeline: Seeding cells on Day (D) 0. D1: Inducing differentiation. D5 observing fusion of myoblast cells. D10: Screening for myo-fiber contraction in cultures that were not stained for analysis; observing polynucleated fibers but no myofiber contractions. D14 Fixing cells and fusion-index-analysis.

FIG. 5C: Day 14: Fixation and fusion-index-analysis based upon staining for myosin heavy chain (MHC) (red) and nuclei (DNA) (shown in blue).

FIG. 5D: Shows a schematic illustration of multinucleated myofibers in MHC (red) and nuclei (DNA) (blue).

FIG. 6A-6G: shows Human Skeletal Myoblast-Derived Poly-Nucleated Fibers growing is micro fluidic chips where Sulfo-SANPAH cross-linked ECM enables formation of almost 2-fold more MHC positive multinucleated fibers.

FIG. 6A-6D: show fluorescent micrographs of immunostained myosin heavy chain (MHC) (red) myo-fibers and DAPI stained nuclei (DNA) (shown in blue) comparing cultures started at the 2 different densities (FIGS. 6A-B: 5×10⁶/ml cells and FIGS. 6C-6D: 1.6×10⁶/ml cells) with and without cross-lined (X-link) ECM-Laminin (Lam).

FIGS. 6E-6F: show phase contrast micrographs of Day 14 cells grown on Laminin (Lam) and cross-linked (X Link) ECM-Laminin (Lam), respectively. More MHC positive multinucleated fibers are observed with X-Linked Laminin after 14 days. White arrows point to 2 exemplary multinucleated myotubes

FIG. 6G shows a graph comparing number MHC+ myo-fibers to the treatments shown in FIGS. 6A-6D where at both cell densities the number of myofibers growing on x-Linked ECM is almost 2-fold more than fibers grown on regular, non-cross-linked, ECM.

FIG. 7A-7F: shows Human Skeletal Myoblast-Derived Poly-Nucleated Fibers growing in microfluidic chips comparing non-cross-linked to cross-linked ECM (Laminin) where more nuclei per myo-tubes are observed growing on cross-linked ECM.

FIG. 7A-7D: show fluorescent micrographs of immunostained myosin heavy chain (MHC) (red) myo-fibers and DAPI stained nuclei (DNA) (shown in blue) comparing cultures started at the 2 different densities with inserts showing higher magnifications of presumptive myo-fibers for each treatment.

FIGS. 7A-7B: 5×10⁶/ml cells and FIGS. 6C-D: 1.6×10⁶/ml cells) with Laminin (Lam) and with cross-linked (X-linked) Laminin-ECM.

FIGS. 7E-7F: Show a 3-fold higher number of nuclei in MHC myo-fibers seeded on exemplary Sulfo-SANPAH cross-linked ECM by graphical comparisons.

FIG. 7E: shows a graph comparing DAPI+ nuclei per MHC+ fiber for determining myo-fiber at the 4 treatments shown.

FIG. 7F: shows a graph comparing percentage of total DAPI+ per channel, i.e. percentage of DAPI in myo-fibers at the 4 treatments shown in FIG. 7A-D.

FIG. 8A-8D: shows one embodiment of a Human iPS-Derived MN and Muscle Cell Co-Culture in-a Tall Channel Microfluidic Chip.

FIG. 8A: shows a picture of a tall channel microfluidic chip (16) in one embodiment seeded with MNs at day 12 of culture into the port (2) of the upper (blue) channel (thick arrow) (1) and human skeletal muscle cells into the port (3) of the lower (red) channel (1) at the end of the other channel (thin arrow). The arrowhead points to a vacuum chamber (4), for optional use.

FIG. 8B: shows iPSC-derived MNs seeded into the upper channel forming a neural network stained with TUJ1 (green); Islet1 (ISL1) (blue); indicating early motor neurons, and Islet1 (ISL1) (blue); HoxB9 (red); indicating more mature motor neurons, while the third frame is a superimposed image showing both early and more mature motor neurons.

FIG. 8C: shows skeletal muscle cells seeded into the lower channel stained with myosin heavy chain (MHC) (green) with an insert showing myofibers at a higher magnification; α-bungarotoxin BTX (pre-BTX) (red), for identifying AchR in the motor end plate, with an insert showing stained cells at a higher magnification; and DNA in nuclei stained then fluoresced in the blue range, with an insert showing myofibers (green) at a higher magnification with unstained regions that likely correspond to multinuclear areas in the myofibers; and

FIG. 8D: shows a schematic illustration of a vertical cross section of a tall channel microfluidic chip where MNs from a Day 12 culture seeded onto the chip develop cell bodies containing nuclei (purple circles), axons and terminal areas next to the membrane separating the top from the bottom channel containing human skeletal muscle cells growing around the edge of the channel.

FIG. 9A-9C: Shows one embodiment of a Human iPS-Derived MN and Muscle Cell Co-Culture in-a microfluidic Chip.

FIG. 9A is a picture of an exemplary microfluidic chip where day 12 MNs are seeded into the top (upper-blue) channel and hSkMCs are in the bottom (lower-red) channel;

FIG. 9B shows a schematic illustration of an exemplary cross section of NMJ microfluidic chip with day 12 MNs in the top channel and hSkMCs in the bottom channel with 3 sets of Experimental Chips for comparing cell densities at the time of seeding: Chip 1: top: 3×10⁶/ml diMN cells and bottom: 5×10⁶/ml hSkMC cells; Chip 2: top: 3×10⁶/ml diMN cells and bottom: 10×10⁶/ml hSkMC cells; and Chip 3: top; 3×10⁶/ml diMN cells and bottom; 20×10⁶/ml hSkMC cells.

FIG. 9C: shows a schematic illustration of a timeline showing co-culture of hSkMCs seeded Day (D) 0 with differentiation (diff) initiated on D1, Day 12 MNs seeded D1, Myofiber formation on D5, myofiber contractions observed D10, a loss of myofibers observed on D11, with fixation and analysis by ICC on D14.

FIG. 10A-10B: shows one embodiment of an experimental system (Experiment 1) as a schematic illustration for testing medium to reduce spontaneous contractions of cells in the microfluidic tall channel chip. Experimental Groups 1-3 directly compare medium harvested from diMNs/hSkMC cultures with coM media in chips containing induced motor neurons (diMNs: Motor-neuron-on Chip) and human Skeletal Muscle Cells (hSkMCs-on-Chip), each cell type growing alone on chips then combined in the same chip in the same media (upper and lower channel) for providing a neuronal-muscular-junction (NMJ-on-Chip).

FIG. 10A: Group 1: shows a schematic illustration of the tall channel chip, with vacuum chambers (4), diMNs in the top channel but no cells in the bottom channel. Group 2: shows a schematic illustration of the tall channel chip with no cells in the top channel but with hSkMCs in the bottom channel. Group 3: shows a schematic illustration of the tall channel chip with diMNs in the top channel and hSkMCs in the bottom channel for providing a NMJ-on-Chip.

FIG. 10B: shows a schematic illustration of cells numbers and media used for growing cells: Group 1: Top: 3×10⁶ diMNs Bottom: none. Group 2: Top: none. Bottom: 10×10⁶ hSkMCs. Group 3: Top: 3×10⁶ diMNs. Bottom: 20×10⁶ hSkMCs.

FIG. 11A-11C: Shows human skeletal muscle cells (hSkMCs) forming myofibers within 8 days post seeding (co-cultures) having spontaneous myo-tube contractions at Day (D) 10 culture that are reduced by using coal culture medium in a microfluidic chip.

FIG. 11A: shows micrographs of hSkMCs growing in chips. White arrows in the magnified region point to multinucleated muscle cell fibers, of which there appears to be more nuclei per fiber in the coM medium;

FIG. 11B: shows micrographs of diMNs growing in chips; and

FIG. 11C: shows micrographs of shSkMCs/diMNs grown in MN/hSkMCs media (upper row of micrographs) and coM medium (lower row of micrographs) growing in chips. Spontaneous myo-tube contraction was observed only in diMNs/hSkMC co-cultures. White arrows in the magnified region point to contacts of MN with a muscle cell fiber.

Inserts show higher magnified areas of cells outlined in the white box for each micrograph.

FIG. 12A-12D: Shows human skeletal muscle cells (hSkMCs) as myofibers with spontaneous myotube contractions at Day (D) 10 (Experiment 3).

FIG. 12A: shows a micrograph of hSkMCs as myotubes growing on top of a membrane of the microfluidic chip in coM media.

FIG. 12B: shows a graph comparing contractions per minute for a myofiber contraction frequency with an average of fibers in two experiments (Experiment 1 and 3) that were combined for a total estimation of myofiber contraction frequency.

FIG. 12C: shows a graph comparing contractions per minute for myofibers having an increased myofiber contraction frequency of myotubes grown on cross linked Laminin ECM over non-cross-linked Laminin covered surfaces.

FIG. 12D: shows a graph comparing contractions per minute for myofibers grown in regular media compared to a culture grown in coM media. When cultured in coM, contraction frequency is around 25% less compared to regular medium conditions.

FIG. 13A-13B: shows schematic illustrations of experimental timelines for comparing co-cultures of hSkMCs with MNs, with and without coM media.

FIG. 13A: shows a schematic illustration of a timeline and cell densities for Group 1 and Group 2 in coM: hSkMCs seeded at 5×10⁶/ml cells and MNs seeded at 3×10⁶/ml cells. hSkMCs seeded Day (D) 0 with differentiation (diff) initiated on D1, Day 12 MNs seeded D1 (as one example 18 h later), D5 formation of myotubes & medium switch to coM, no myofiber contractions observed D10, no loss of myofibers observed on D12, fixation and analysis by ICC on D14, duplicate chips on D20 showed no loss of myofibers.

FIG. 13B: shows a schematic illustration of a timeline and cell densities for Group 3: hSkMCs seeded with MNs: Day 0: seeding hSkMCs; Day 1: (18 h later) seeded diMNs (d12); Day 5: formation of myotubes, no medium switch; Day 10; observation of myofiber contraction; Day 11: observing progressive loss of myofibers; Day 14: fixation and analysis by ICC; in chip cultures left to D20, there is almost a complete loss of myofibers.

FIG. 14A-14B: Shows schematic illustrations of embodiments of a microfluidic device.

FIG. 14A: is a schematic illustration showing one embodiment of the microfluidic device or chip (16), comprising two microchannels (1), each with an inlet and outlet port for the upper channel (2) and lower channel (3), as well as (optional) vacuum ports (4).

FIG. 14B is a topside schematic of an embodiment of the perfusion disposable or “pod” (10) featuring the transparent (or translucent) cover (11) over the reservoirs (12), with the chip (16) inserted in the carrier (17). The chip can be seeded with cells and then placed in a carrier for insertion into the perfusion disposable or pod, whereupon culture media in the reservoirs flows into the microchannels and perfuses the cells (e.g. both MNs and hSMCs).

FIG. 15A-15B: Shows schematic illustrations showing one embodiment of microfluidic devices, including for providing an “air dam” for isolating one channel.

FIG. 15A: is a schematic illustration showing one embodiment of a microfluidic device or chip (16) (viewed from above), the device comprises top (apical; dotted line) and bottom (basal; solid line) channels. As an example, motor neurons are seeded into the upper (apical) channel and human skeletal muscle cells are seeded into the lower (basal) channel. In one embodiment, an “air dam” is created for part of a protocol, described below, where the two Xs are indicating that channels are blocked during at least part of the protocol.

FIG. 15B: is a schematic illustration showing one embodiment of how ports, upper (2) and lower (3) of a microfluidic device or chip (16) can be utilized to deposit fluids carrying surface coatings (e.g. dissolved proteins) and/or seed the cells using pipette tips. This image, in part, shows one embodiment of a modification to the typical chip ECM coating protocol based on the need in some embodiments to coat the top and/or bottom channels with different ECM solutions in wet and/or dry conditions.

FIG. 16A-16B: shows schematic illustrations of tall channel microfluidic NMJ-on-chip with one embodiment of an experimental timeline (Experiment 4) set up and time course for comparing co-cultures of hSkMCs with MNs under flow for longer culture times.

FIG. 16A: shows a schematic illustration of a tall channel microfluidic chip, from left to right, view of vertical 2-channel chip (i.e. the top channel is above the bottom channel as shown in Stage 1, with hSkMCs covering the entire surface of the bottom channel, and Stage 2 with diMNs seeded into the top channel.

FIG. 16B: shows a schematic illustration of one embodiment of a timeline where hSkMCs are seeded Day (D) 0 with differentiation (diff) initiated on D1, D5: formation of myotubes & medium switch to coM media, then Day 7-10: no myofiber contraction, on Day 20 start muscle cells under flow at 10 ul/hour, continued to D29 when flow is stopped. Day 30: seed diMNs (d12) (not in coM media for observing baseline contractions). Day 37: myotubes are spontaneously contracting: fixation and analysis (including ICC).

FIG. 17A-17G: shows an exemplary co-localization study of iPS-Derived MNs and Muscle Cells showing formation of NMJs between diMNs and hSkMCs (Experiment 4). Cells were stained with α-bungarotoxin (BTX) for identifying suggestive NMJ areas where motor end plate (green), neurons are stained with Tubulin beta-3 chain (Tubb3) (red) and muscle myosin heavy chain (MHC) (blue) were fluorescently imaged on individual channels then merged. The blue channel of MHC staining is not shown in FIG. 17A-17D.

FIG. 17A: shows a low power fluorescent micrograph where Tubb3 (red) neuronal staining shows neurite extension along myotubes with oval areas (green) suggestive of lower motor nerve termini whose distribution over a myotube suggests motor end plates.

FIG. 17B-G: shows higher power fluorescent micrographs of the suggestive NMJ areas (white arrows) are identified by superimposed staining i.e. co-localization, where the red stained nerve terminal neuron bulb is co-localized with BTX green staining of motor end plates producing a yellow NMJ.

FIG. 17E-17G: The blue channel of MHC staining is shown showing a MHC containing muscle fiber at the yellow stained NMJ.

FIG. 18A-18D: shows florescent micrographs of stained cells in a microfluidic chip. Co-Localization Study of iPS-Derived MNs and Muscle Cells. Both diMNs and hSkMCs are in close proximity to each other as determined from initial ICC analysis and 3D reconstruction of confocal microscope images (i.e. combined z-stacks). A partial loss of myotubes were observed due to lack of ECM stability

FIG. 18A and FIG. 18B: α-bungarotoxin (BTX) for identifying the motor end plate (green), skeletal muscle marker, desmin, (red) and DNA (DAPI) (shown in blue). The red muscle fiber is multinucleated with numerous green motor end plates.

FIG. 18B: a higher magnification of FIG. 18A, 3 white arrows point to co-localization of α-bungarotoxin (BTX) for identifying the motor end plate (green) and skeletal muscle marker, desmin, (red) as olive, white dark orange areas depending upon concentration of stain.

FIG. 18C and FIG. 18D: motor end plate (green) BTX and neurofilament H non-phosphorylated (SMI 32) (red) and DNA (DAPI) (shown in blue).

FIG. 18D: a higher magnification of FIG. 18C, 3 white arrows point to co-localization of a motor end plate (green) BTX, neurofilament H non-phosphorylated (SMI 32) (red) as olive-white areas depending upon concentration of stain.

FIG. 19A-19B: shows schematic illustrations of one embodiment of experimental timelines for using NMJ-on-chips (Experiment 5) as a set up and time course for using co-cultures of hSkMCs with MNs for live imaging and pharmacology studies.

FIG. 19A: shows a schematic illustration of a tall channel microfluidic chip, seeded with hSkMCs at Day 0 (D0) in the bottom channel, culting up to D9, without observing muscle contractions, then D9 seeding diMNs (d12). In one embodiment only in Group 2. In some embodiments, more than one group of hSkMCs receive MNs. On days 15, 16 and/or 17. live imaging of pharmacology assays are done as shown schematically, for one example, in FIG. 19B.

FIG. 19B: shows a schematic illustration of one embodiment of a timeline where a NMJ-On-Chip with spontaneous contracting muscle fibers is used for a pharmacology study, i.e. testing agents for inducing or reducing muscle contractions on a baseline chip with or without spontaneously contracting myofibers, in one embodiment, treating NMJ chip with 75 uM Glutamine (Glut) in the NM (upper) channel), in one embodiment, treating NMJ chip with 12 uM alpha-turbocurarine in the hSkMC (lower) channel), in one embodiment, washing out alpha-turbocurarine, in one embodiment, treating NMJ chip with 100 uM Glutamine (Glut) in the NM (upper) channel).

FIG. 20A-20B: Shows exemplary High Content Imaging as immunohistochemistry of iPSC derived Myo-fibers, on fixed cells (Experiment 5).

FIG. 20A: shows a fluorescent micrograph of the entire width and length of immunostained cells in a microfluidic NMJ chip. α-bungarotoxin BTX (green), Neuron-specific Class III β-tubulin (TuJ1) (red) and myosin heavy chain (MHC) (blue).

FIG. 20B: shows a higher power fluorescent micrograph of the channel in the chip shown in FIG. 20A.

FIG. 21A-21B: shows micrographs of cells grown as shown in Experiment 5 for pharmacology and in-chip imaging for NMJ-On-Chip.

FIG. 21A: shows phase contrast micrographs of myotubes and neurons in chips, higher magnified areas are shown below the larger micrograph white arrows point to potential NMJs where myotubes are adjacent to neurons.

FIG. 21B: shows fluorescent micrographs of superimposed (co-localized images) of neurons stained with a neuronal microtubule marker, Tau, (green) a microtubule stabilization protein, for identifying neurons and motor end plates with BTX (red) (labeling AChRs) for identifying NMJs, where neuronal branches co-localize with end plates. Smaller micrographs show higher magnified areas outlined by corresponding white boxes. White arrows point to motor end plates of myotubes, some of which are in close proximity to neuronal axons.

FIG. 22AA-22CC: shows an exemplary method of growing motor neurons in a microfluidic chip where the MN cells of neural networks have spontaneous calcium bursts.

FIG. 22AA: shows a microfluidic chip seeded with MNs at day 12 of culture.

FIG. 22BB: shows an exemplary timeline where MN precursor cells from Day 12 cultures are seeded at Day 0 in the microfluidic chip, MN network formation is observed a Day 10 on the chip (Day 18 overall from the start of the original MN culture).

FIG. 22CC: shows exemplary images produced by high content life imaging of cells in chips showing Ca++ imaging of diMN cells on Day 12 after seeding onto the microfluidic chip; at high magnification (20×). diMNs show repetitive calcium bursts as visualized via Flou4 labeling in color within the cellular areas, e.g. cell bodies, axons and terminal bulbs, in neuronal networks, where the concentrations of Ca++ are shown by yellow-lower levels, red-higher than yellow areas and highest levels in white areas within the red areas, as shown in the neuron cell bodies.

FIG. 22A: shows exemplary Ca++ imaging of FIG. 22CC in black and white, where the highest amounts of Ca++ are white areas in black and white micrographs, white arrowheads point to cellular areas with concentrated Ca++.

FIG. 22B: Shows a higher magnification of a cell in the center of the micrograph in FIG. 22CC/FIG. 22A with two white arrowhead markers used to identify the same area through the different planes of focus.

FIGS. 22D-22J: shows exemplary Ca++ imaging in color from confocal high content micrograph z-stack layers through the cell (shown in FIG. 22B) where higher concentrations of Ca++ are shown by yellow/red/white areas in the neuronal cytoplasm, which discharge and recharge then discharge over time. White arrowheads mark the same location of the cell shown in FIG. 22B-FIG. 22J.

FIG. 22K: shows a graph of average intensity of Ca++ vs. elapsed time (seconds).

FIG. 23A-23B: shows exemplary fluorescent micrographs of NMJ-On-Chips using iPSC derived Myo-fibers (iSKMCs) as superimposed (co-localized images) of neurons and myotubes.

FIG. 23A: shows a fluorescent micrograph of nerve axons (red) parallel to multinucleated (blue) muscle heavy chains within muscle myofibers (green) showing separation between internal myosin and external nerve fibers. Myosin (MHC: myosin heavy chain) (green), neuronal nerve fibers TuJ1 (red) and DNA (DAPI) (shown in blue)

FIG. 23B: shows a fluorescent micrograph view on end (as compared to the orientation in FIG. 23A) for a different view, i.e. x-z image, of muscle Myogenin (green), nerve TuJ1 (red) and DNA (DAPI) (shown in blue) where nuclei superimposed on the muscle staining shows light blue, see example at the white arrow.

### DESCRIPTION OF THE INVENTION

The invention relates to culturing motor neuron cells together with skeletal muscle cells in a fluidic device under conditions whereby the interaction of these cells mimic the structure and function of the neuromuscular junction (NMJ) providing a NMJ-on-chip. Good viability, formation of myo-fibers and function of skeletal muscle cells on fluidic chips allow for measurements of muscle cell contractions. Embodiments of motor neurons co-cultures with contractile myo-fibers are contemplated for use with modeling diseases affecting NMJs, e.g. Amyotrophic lateral sclerosis (ALS).

In one embodiment, the present invention contemplates a NMS-on-chip where at least one population of cells is derived from a patient diagnosed with a disorder of the nervous system. While it is not intended that the present invention be limited to a particular CNS disorder, in one embodiment, the disorder is ALS. Amyotrophic lateral sclerosis (ALS) is a severe neurodegenerative condition characterized by loss of motor neurons in the brain and spinal cord. In one embodiment, the present invention contemplates generating induced pluripotent stem cells (iPSCs) from patients with ALS and differentiating them into motor neurons progenitors and/or skeletal cell progenitors for seeding on a microfluidic device. Patients with ALS have progressive deterioration of the neurons, alterations of skeletal muscle fibres are observed in patients with ALS, including but not limited to accumulation of abnormal protein inclusions, mitochondrial changes, skeletal muscle atrophy, etc. There are currently no effective treatments for ALS. In one embodiment, the present invention contemplates the NMJ-on-chip as a model system for testing drugs so as to predict success in subsequent clinical trials.

In other embodiments, diseases where skeletal muscle abnormalities are found include multiple system atrophy.

It is contemplated that iPSC technology can be used together with microfluidic chips to mimic patient-specific phenotypes in disease states. Thus, in one embodiment, iMNs are derived from a patient diagnosed with or at risk for a disease. In one embodiment, ihSkMCs are derived from a patient diagnosed with or at risk for a disease. In yet another embodiment, the iMNs and ihSkMCs are generated from the same patient line, e.g. the same patient stem cells. In one embodiment, the patient has symptoms of a CNS disorder, and more specifically, a neurodegenerative disease. In one embodiment, the neurodegenerative disease is ALS.

More specifically, the embodiments described herein show that functional NMJ-on-Chip, i.e. NMJ-on-chip (diMNs/hSkMCs) with reduced spontaneous muscle contractions, are superior over co-cultures (2D) of MN and muscle cells. Further, hSkMCs (human skeletal muscle cells) grown on microfluidic chips as described herein, i.e. SkMCs-on-chip, are superior over plate cultures of muscle cells.

In particular, NMJ-on-Chip, in one embodiment, comprises a motor neuron-on-chip, e.g. patient iPSC-derived MNs, expressing neuronal markers, are combined with a human skeletal muscle-on-chip: containing contractile tissue. Although co-culture of muscle and neuronal cells on a tall channel microfluidic chip was successful, it was determined that to provide a more robust and functional NMJ-on-chip there was an apparent need to inhibit spontaneous muscle fiber contractions induced by co-culture with MNs. In part, because by adding medium, or blockers to the culture medium, for reducing generation of an action potential (AP) in the NMJ, there was a lower loss of myotubes over time. In other words, human skeletal cells co-cultured with human MNs showed spontaneous muscle fiber contractions resulting in a loss of myotube structure beginning within 24-48 hours. By switching to a medium that reduces spontaneous contractions the myotubes remain viable longer over time. Further, reduction of spontaneous contractions allows the controlled addition of pharmacology agents on older co-cultures. In contrast, in cultures of muscle cells without neurons there was little spontaneous twitching, i.e. contractions, and these cultures remained viable over longer time periods.

In summary, a Human Muscle Cell Culture in-Chip was first developed in a single channel (Quad) chips, HSkMCs were seeded into an upper channel at 2 different cell densities; differentiation was induced then muscle cells were screened for myo-fiber contraction. It was observed that human skeletal myoblast (hSkMCs) differentiate into poly-nucleated myofibers (d5) with spontaneous myofiber contractions (d10). Secondly, hSkMCs were seeded into the lower channel of a 2-channel microfluidic chip, including a tall chip.

A NMJ-on-chip was provided by combining the 2 chips, i.e. human iPS-derived MN and skeletal muscle cell-on-chip. hSkMCs were seeded into the lower channel of a tall channel chip, then diMNs (day 12) were added to the upper channel. Medium optimization was done in order to reduce spontaneous contractions in chips with diMNs & hSkMCs.

Thus, exemplary steps for providing a functional NMJ-on-Chip by combining motor-neurons on a chip (upper blue channel) with skeletal muscle cells on a chip (lower-red) channel include: Seeding the bottom (lower-blue) channel as a skeletal muscle-on-chip capable of producing contractile muscle tissue expressing markers myosin heavy chain (MHC) (green), pre-BTX (α-bungarotoxin) (red) identified by immunohistochemistry and stained for DNA (blue) shown by fluorescent microscopy. Seeding the upper channel of the microfluidic chip with patient iPSC-derived MNs that under chip culture conditions will express neuronal expressing markers Neuron-specific Class III β-tubulin (TuJ1) (red), selectivity/selective factor 1 complex (for RNA polymerase) (SL1) (blue), homeobox B9 (HOXB9) (red), identified by immunohistochemistry (IHC) as shown by fluorescent microscopy. In some embodiments, spontaneous contractions may be stopped by adding calcium channel blockers or sodium channel blockers to the culture media.

Several embodiments for experiments were provided, along with exemplary results. For examples, Experiment (Exp) 1 showed that hSkMC seeding density at 3×10⁶ cells/ml, but loss of cells 24 h after contracting activity. Experiment 2 showed that Sulfo-SANPAH cross-linked ECM provides more stability to hSkMCs. Experiment 3 showed improved hSkMCs in-chip integrity. However this was lost 48 h after contraction activity. Experiment 4 showed that hSkMC integrity in chip is expandable over time (in monoculture). Experiment 5 showed that pharmacology and imaging was possible for measuring functional NMJ interactions. Thus, in some embodiments, pharmacological testing of agents for treating diseases, such as ALS NMJs, is contemplated. Including using cells derived from ALS patients.

Additionally, contemplative embodiments include, but are not limited to increasing cell in-chip longevity; anchoring hSkMCs; further reducing spontaneous activity of neurons and/or NMJs; changing cell separation, for example, increasing and/or decreasing pore size of the membrane.

### I. The Neuromuscular Junction.

The Neuromuscular Junction (NMJ) refers to the interface between spinal motor neurons and skeletal muscle cells. As each myelinated motor axon reaches its target muscle, it may divide into 20-100 unmyelinated terminal fibers where each terminal fiber innervates a single muscle fiber. The combination of the terminal fibers from a motor axon and the muscle fibers they serve is called a motor unit. The terminal fibers have contain both potassium (K+) and sodium (Na+) channels, which control the duration and amplitude of the action potential. In contrast, the nerve terminals, i.e. multiple synaptic end bulbs of each terminal fiber, have a paucity of Na+ channels and the action potential continues passively into this area. The nerve terminal contains synaptic vesicles (SVs), each of which contains approximately 5000-10,000 molecules of the neurotransmitter acetylcholine (ACh).

The mature NMJ can be divided into presynaptic, synaptic, and postsynaptic phases. The following sections describe components and function of NMJs for reference.

A. In Vivo Components of the NMJ.

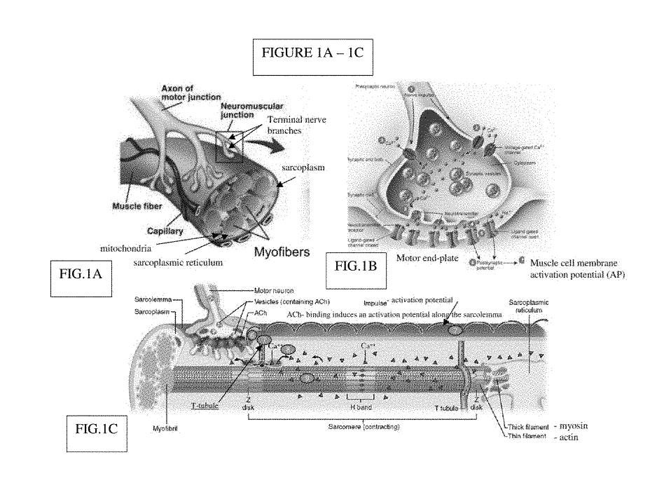

FIG. 1A: shows a schematic illustration of the exterior of neuromuscular junctions where the yellow axon of a motor nerve at the motor junction has non-myelinated terminal nerve branches forming neuromuscular junctions (one example of an NMJ is outlined by a square). The neuronal terminal nerve branches have synaptic end bulbs (see FIG. 1B) located opposite of a muscular fiber end plate (see FIG. 1B). FIG. 1A also shows a schematic of an interior view of a muscle fiber composed of numerous myo-fibers interspersed with mitochondria (blue), sarcoplasmic reticulum (yellow tubes) within the sarcoplasm of a muscle fiber cell (myocyte).

B. In Vivo Neuronal Induction of an Action Potential (AP).

FIG. 1B: shows a cut-out schematic illustration of the interface between spinal motor neurons and skeletal muscle cells, e.g., a NMJ, for demonstrating the steps of normal motor neuronal activation of muscle fibers. Step 1) An action potential of a myelinated axon reaches the non-myelinated axon terminal branch. Step 2) Voltage-dependent calcium gates open allow Ca++ to enter the end bulb which in Step 3) induces the movement of neurotransmitter containing vesicles to merge with the cell membrane at the end of the synaptic bulb opposite muscle cell acetylcholine (ACh) receptors located in the motor end plates. Neurotransmitter vesicles containing acetylcholine (ACh) are emptied (by exocytosis) into the synaptic cleft. Step 4) Freed ACh from the vesicles then diffuses across the cleft to bind to postsynaptic receptors on the sarcolemma of the muscle fiber in the motor end plate area. Step 5) This ACh binding causes ion channel pumps to open which allows sodium ions to flow across the membrane into the muscle cell while fewer K+ ions are transported out of the cell i.e. (3) Na+ ions enter the cell cytoplasm while (2) K+ ions are transported out, thus triggering a post synaptic action potential (end plate potential) in the NMJ, i.e. the end plate of the muscle sarcolemma. Step 6) the postsynaptic action potential (AP) generated at the end plate, Step 7) AP wave, i.e., sarcolemma membrane depolarization, travels across the muscle cell membrane.

Not shown in FIG. 1A-1C, neuron-neuron activations occur when 1N) The axon action potential across an axon reaches the axon terminal. Step 2N) Voltage-dependent calcium gates in the synaptic end bulb open allowing Ca++ to enter the terminal branch which induces the movement of neurotransmitter containing vesicles to merge with the cell membrane at the end of the synaptic bulb opposite the dendrites of an adjacent neuron. Step 3N) Neurotransmitter vesicles containing acetylcholine (ACh) are emptied (by exocytosis) into the synaptic cleft, i.e. the fluidic space in between the cells. Step 4N) Freed ACh from the vesicles then diffuses across the cleft to bind to postsynaptic receptors on the dendrites. Step 5N) This ACh binding causes ion channel pumps to open which allows sodium ions to flow across the membrane into the neuronal cell while fewer K+ ions are transported out of the cell, thus triggering a postsynaptic action potential in the dendrites of the receiving neuron which travels to across the cell membrane to the opposite axon terminal end for triggering an AP in the next cell, starting a Step N1.

C. In Vivo Neuronal Induction of Skeletal Muscle Contraction as a Myofiber (Myotube) Contraction.

FIG. 1C: shows a schematic illustration of a muscle cell (myocyte) depicting how the postsynaptic action potential (AP), triggered by the NMJ, in the sarcolemma of the motor end plate, in Step 6) travels to nearby areas of the T-tubules (i.e. a wave of ion pump activation that travels along the membrane whereby (3) Na+ ions enter the cell cytoplasm while (2) K+ ions are transported out of the cell cytoplasm. Further in Step 7) When the AP reaches areas of the T-tubule portion of the sarcolemma, destabilizing this area of the membrane, the AP in the sarcolemma of the T-tubule area causes the T-tubule to induce the release of Ca++ from the sarcoplasmic reticulum. Step 8) The destabilized sarcolemma then triggers a wave of Ca++ release across the sarcoplasmic reticulum membrane inside of the myocyte. Step 9) The rise in intracellular Ca++activates contraction of myofibrils, i.e. myosin-actin interactions.

After Ach activates the ion pump, it diffuses away to be broken down by endogenous Acetylcholinesterase (ACHE), i.e. inactivates Ach.

D. Plate Co-cultures of Motor Neurons with Skeletal Muscle Cells.

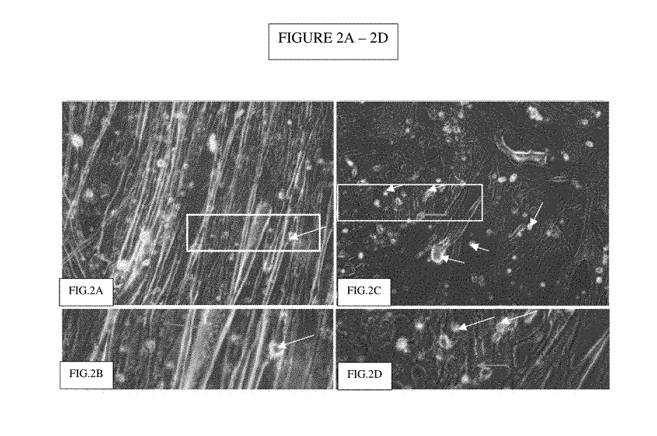

Attempts were made to provide NMJs by co-culturing Motor Neurons (diMN) with human Skeletal Muscle Cells (hSkMCs) in 2 dimensional (2D) plate cultures. Individual cultures of muscle cells showed formation of some multinucleated myotubes (see, FIG. 2A and FIG. 2B), and co-cultures of hSkMCs with diMNs resulted in an occasional potential NMJ where the neurons grew on top of the myotubes. However, the majority of cells appeared unhealthy and possibly dying (see, FIG. 2C and FIG. 2D). These micrographs of static co-cultures were taken on day 37.

FIG. 2A-2D shows 2-Dimensional (2D) motor neurons (MN) and muscle cell co-cultures grown in static plates, on day 37 of culture.

FIG. 2A: shows a micrograph of healthy human muscle skeletal cells (hSkMCs);

FIG. 2B: shows a higher magnification of cells in FIG. 2A, where the green arrow points to one exemplary multi-nucleated myotube;

FIG. 2C: shows a micrograph of a co-culture of direct induced motor neurons (diMNs) on top of hSkMCs where white arrows point to rounded cell bodies, a green arrow points to an exemplary myotube and a red arrow points to an exemplary neuron on top of said myotube; and

FIG. 2D: shows a higher magnification of cells in FIG. 2C where the red arrow points to neuronal branches on top of a myotube identified by a green arrow. White boxes outline the areas shown in higher magnification.

Therefore, there is a need for providing more viable co-cultures of MN and hSkMCs for providing numerous functional NMJs.

### II. Generation of Motor Neurons for Providing Embodiments of a NMJ-on-Chip.

A. Neuronal Cells.

In this example, several exemplary embodiments are provided for the generation of motor neurons is provided using iPSCs as the starting material, see, Table 1 and Table 2 for exemplary concentrations and timelines. In one embodiment, a MN-on-chip is provided with MNs seeded into the upper channel of a microfluidic chip. In another embodiment, MNs are seeded into the upper channel of a NMJ-On-Chip.

Cells are prepared either directly from cultured iPSCs or from frozen lots of pre-differentiated cells. Cells are thawed (or dissociated fresh) and seeded into the chip at day 12 (in the case of iMN differentiation) and at various points in neural differentiation. See, Table 1 for one embodiment for preparing iMN cells.

As another embodiment, iPSC-derived forebrain neural progenitor cultures (dubbed EZs) were cultured in chip either dissociated or as neural spheres that attached and extended in 3 dimensions.

More specifically, MNs, for example, cells are seeded into microfluidic chips at day 12 of differentiation either from freshly differentiated cultures or directly from a thawed vial.

Conditions were tested for seeding neural (EZ spheres and iMNPs) from frozen stocks of cells on surfaces treated with different extracellular matrices (ECMs). While frozen stocks of cells can be used (particular for the neural cells), it was found that better results can be obtained when fresh cells are used for seeding chips.

As another embodiment, Schwann cells, as precursors or mature cells, may be added to provide a mylin sheath for MNs. In some embodiments, Schwann cells are derived from patient cells, such as patients having a neuromuscular disease.

Culture of these cells in a microfluidic device, such as a microfluidic chip with flow as herein described, whether alone or in combination with other cells, drives maturation and/or differentiation further than existing systems. For example, a mature electrophysiology of the neurons includes negative sodium channel current, positive potassium channel current, and/or action potential spikes of amplitude, duration and frequency similar to neurons in a physiological environment or when compared to static culture neurons, static culture neurons lack one or more of the aforementioned features.

Observed characteristics of the in vitro “NMJ-on-chip” of the present invention include: (1) neuronal networks comprising motor neurons; (2) optional cell-to-cell communication between neurons exemplified by contact of the neuronal dendrites with neuronal terminal bulbs; (3) optional extended neurite projections exemplified by contact of the neuronal terminal bulbs with muscle cells (e.g. terminal bulb contact by partial transmigration of the membrane separating these cells); (4) optional fluid flow that influences cell differentiation and neuronal muscular junction formation; and (5) high electrical resistance representing the maturity and integrity of the NMJ components.

With respect to skeletal muscle cells, in one embodiment, the present invention contemplates hSkMCs which form a lumen on the chip (for example, completely lining the bottom, sides and top of a flow channel, at least for a portion of its length). Among other advantage (e.g. hSkMCs layer stability) this potentially enables the use of the device with blood or blood components. With respect to selective permeability, the present invention contemplates, in one embodiment, introducing substances in a channel with the hSkMCs such that at least one substance passes through the membrane (e.g., hSkMCs on the bottom side of the membrane) and into a channel above the membrane, and detecting said at least one substance (e.g. with antibodies, mass spec, etc.).

Although there is a strong need for a model of the human neuronal muscular junction, it is also desirable to develop models of NMJs of other organisms (not limited to animals). Of particular interest are models of, for example, mouse, rat, dog, and monkey, as those are typically used in drug development. Accordingly, the neuronal muscular junction: NMJ-on-chip can make advantage of not only human-derived cells but also cells from other organisms. Moreover, although it is preferable that all cell types used originate from the same species (for example, in order to ensure that cell-cell communication is effective), it may be desirable at time to mix species (for example, if a desired cell type is scarce or possess technical challenges).

B. Exemplary Timeline.

C. Optional Neuropatterns.

With respect to neurite projections, in one embodiment, the present invention contemplates seeding on nanopatterned surfaces which promote extended and direct (e.g. along a relatively linear path) neurite growth. The preferred nanopattern is linear valleys and ridges, but alternatives such as circular, curved, or any other desired shape or combination thereof are also contemplated.

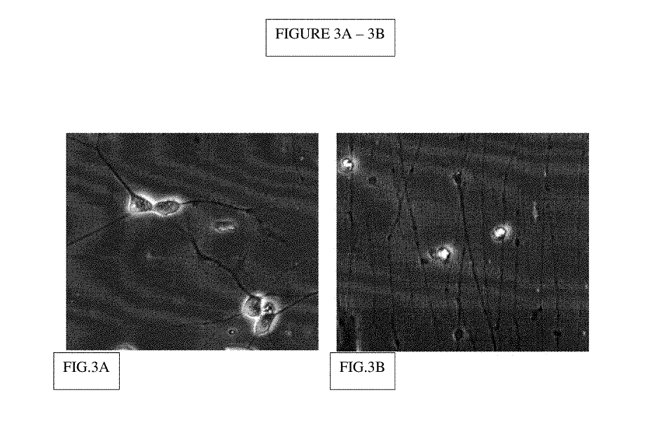

Thus, the present invention contemplates, in one embodiment, utilizing nanopatterned surfaces for seeding cells. FIG. 3A-3B shows a first image (FIG. 3A) where iMNPs were seeded on a plain (un-patterned) surface, as well as a second image (FIG. 3B) where the same cells were seeded on a nanopatterned surface, resulting in directed neurite growth. The nanopatterned surface results in directed neurite growth (e.g. in a line pattern). FIG. 3A-3B: shows exemplary phase contrast images for embodiments of neuronal growth. FIG. 3A: shows iMNs seeded on a plain (un-patterned) surface; and FIG. 3B: shows a duplicate sample of cells (as in FIG. 3A) that were seeded on a nanopatterned surface, resulting in directed neurite growth.

Such nanopatterning can be applied to the membrane or any surface of the NMJ-on-chip. In particular embodiments, the nanopatterning is applied to the top surface of the membrane to direct neurite growth for neuron seeded on said surface. It is desired in some uses to direct neurite growth, for example, in studying neuron biology or disease (e.g. conditions that disturb neurite growth or its directionality), as a readout of neuron or NMJ health (e.g. by monitoring neurite growth or its directionality) or in facilitating measurements (e.g. using calcium imaging, IHC or number and/or quality of NMJs, or using a multi-electrode array or patch clamping). The preferred nanopattern is linear valleys and ridges, but alternatives such as circular, curved, or any other desired shape or combination thereof are also contemplated. Linear nanopatterning can include, for example, line spacing ranging from 10 nm to 1 um, 0.5 um to 10 um or 5 um to 50 um, and line depth ranging from 10 nm to 100 nm, 50 nm to 1000 nm, 200 nm to 5 um or 2 um to 50 um.

D. Calcium Flux High Content Imaging.

Calcium (Ca) imaging or imaging using voltage-sensitive dyes or proteins offer similar advantages to electrophysiological readouts but offers the advantage that no electrodes are necessary.

Ca imaging may occur in the presence of calcium or voltage-sensitive dyes or proteins, to allow the potential recording and optional manipulation of neuronal excitations. These measurements can be used, for example, to provide an indication of neuronal maturation or as a readout of neuron health. Accordingly, some aspects of the present invention include methods of measuring spontaneous, or induced by adding an agent, neuronal excitation.

In turn, neuronal maturation or health can be used as indicators of NMJ-on-chip quality (for example, before starting an experiment) or as an experimental endpoint indicating, for example, that an agent has affected creation of APs, a disease condition has emerged, the NMJ has been modified or compromised, or conversely, that the NMJ or neural function or health have improved. This type of imaging allows observations of neuronal function in the microfluidic chips in real-time. Thus, in one embodiment, neuronal excitation in NMJ-on-chip induced muscle contractions. In one embodiment, addition of tetrodotoxin (TTX), which is a potent blocker of voltage-gated calcium channels, ablates this activity.

In some embodiments, a photograph showing Ca++ hot spots and changes in Ca++ concentrations is a single fluorescent image from a movie of such images. For one example, a movie comprises z-stacks from confocal microscopy images.

High content imaging refers to imaging fixed or live cells within a chip. In some embodiments, Ca flux assays on neurons are imaged within the cultures growing in chips.

E. Spontaneous Calcium Bursts in MN Networks in-Chip.

Negative sodium channel currents (Na⁺) and positive potassium channel (K⁺) are necessary for normal neuron function and become more pronounced as a neuron matures. In fact, highly complex and repetitive bursts of neuronal activity are indicative of neuronal networks being established in the chip. When induced to fire by injecting current into the neuron at day 6 in chip, more resolved action potentials are observed in these chips as compared to traditional neuronal cultures.

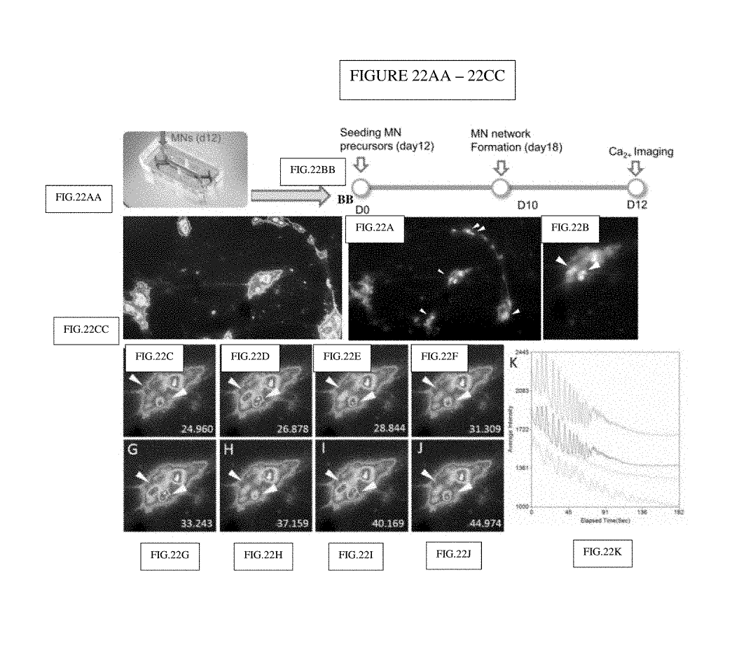

In a controlled study, live cell imaging was performed on diMNs that had been cultured in the chip (MN-on-Chip) (FIGS. 22BB-22J). High content imaging of neuron calcium flux was recorded and plotted with respect to time (FIG. 22K). Calcium flux events or peaks correspond to neural activity and were counted by both automated software and blinded human technician. Each event was assigned a time-stamped value and depicted for each tracked neuron with respect to time. This Calcium (Ca++) flux live cell assay showed Ca flux in relation to spontaneous neuronal activity, i.e. firing. For examples, see FIG. 22AA-22CC.

FIG. 22A-22CC: shows an exemplary method of growing motor neurons in a microfluidic chip where the MN cells of neural networks have spontaneous calcium bursts.

FIG. 22AA: shows a microfluidic chip seeded with MNs at day 12 of culture.

FIG. 22BB: shows an exemplary timeline where MN precursor cells from Day 12 cultures are seeded at Day 0 in the microfluidic chip, MN network formation is observed a Day 10 on the chip (Day 18 overall from the start of the original MN culture).

FIG. 22CC: shows exemplary images produced by high content life imaging of cells in chips showing Ca++ imaging of diMN cells on Day 12 after seeding onto the microfluidic chip; at high magnification (20×). diMNs show repetitive calcium bursts as visualized via Flou4 labeling in color within the cellular areas, e.g., cell bodies, axons and terminal bulbs, in neuronal networks, where the concentrations of Ca++ are shown by yellow-lower levels, red-higher than yellow areas and highest levels in white areas within the red areas, as shown in the neuron cell bodies.

FIG. 22A: shows exemplary Ca++ imaging of FIG. 22CC in black and white, where the highest amounts of Ca++ are white areas in black and white micrographs, white arrowheads point to cellular areas with concentrated Ca++.

FIG. 22B: shows a higher magnification of a cell in the center of the micrograph in FIG. 22CC/FIG. 22A with two white arrowhead markers used to identify the same area through the different planes of focus.

FIGS. 22D-22J: shows exemplary Ca++ imaging in color from confocal high content micrograph z-stack layers through the cell (shown in FIG. 22B) where higher concentrations of Ca++ are shown by yellow/red/white areas in the neuronal cytoplasm, which discharge and recharge then discharge over time. White arrowheads mark the same location of the cell shown in FIG. 22B-FIG. 22J.

FIG. 22K: shows a graph of average intensity of Ca++ vs. elapsed time (seconds).

III. Generation of hSkMCs for Providing Embodiments of a NMJ-on-Chip.

In this example, several exemplary embodiments are provided for the generation of hSkMCs using iPSCs as the starting material. In one embodiment, a hSkMC-on-chip is provided where hSkMCs may be seeded on the upper or the lower channel of the chip. In some embodiments, hSkMCs are seeded and used in quadruple (Quad) single channel chips.

In some embodiments, myoblasts are derived from patient samples for seeding chips. In some embodiments, iPS cells derived from patient cells are used for seeding chips.

As another example, in one embodiment, induced skeletal muscle progenitor cells are derived from induced pluripotent stem cells, but they are not fully differentiated. In one embodiment, induced skeletal muscle progenitor cells are differentiated on-chip to generate multinucleated myotubes, and ultimately mature striated skeletal muscle myotubes.

Thus, in one embodiment, the present invention contemplates a method of culturing cells, comprising: a) providing a microfluidic device (optionally comprising a membrane, said membrane comprising a top surface and a bottom surface); b) seeding induced skeletal muscle progenitor cells (on said bottom surface so as to create seeded cells); c) exposing said seeded cells to a flow of culture media for a period of time (days to weeks to months) under conditions such that said at least a portion of said progenitor cells differentiate into multinucleated myotubes (and preferably wherein said hSkMCs display a mature phenotype based on testing described herein or staining).

A. Human Skeletal Muscle Cells.

Muscle tissue develops from specialized mesodermal cells called myoblasts. Several myoblasts fuse together to form a myotube. Myotubes are immature multinucleated muscle fibers. Myotubes mature into striated skeletal muscle fibers. Satellite cells are found along the outside of the fibers in vivo. Satellite cells refer to precursors to skeletal muscle cells, able to give rise to satellite cells or differentiated skeletal muscle cells. They have the potential to provide additional myonuclei to their parent muscle fiber, or return to a quiescent state.

The following describes exemplary methods, e.g. for differentiating iPSCs, providing a Muscle Cell Culture-on-Chip.

1. Skeletal Muscle Differentiation from Human iPSCS.

The starting density of cells affects the success of skeletal muscle cell differentiation. The starting iPSc density described herein is exemplary for the cell lines described herein. However each IPSC line is different so the optimal density should be determined according to each individual cell line's growth (e.g. doubling) rate. For cell lines shown herein, an exemplary recommended cell density and volume of media: 12 or 24 wells 15,000-18000 cells/cm² and for 96 wells 5000 cells/cm². One embodiment for a method providing human induced pluripotent stem cells (iPSCs) for use in providing induced hSkMCs is described as follows.

Coat plates with ECM, e.g. Matrigel. Add appropriate volume, see e.g. below, in a sterile tissue culture hood. For a 6 well plate—1 mL/well; 24 well plate—250 μL/well; and 96 well plate—50 μL/well. Leave Matrigel in wells for at least 1 hr at room temperature for coating surfaces. Coating may also be done for more than an hour.

For deriving human iPSC (hiPSC) skeletal cell cultures from hiPSCs: Grow and expand iPSC cultures on Matrigel coated surfaces with mTeSR Media supplemented with Rock Inhibitor (Y-27632) (such as from Sigma-Aldrich, St. Louis, Mo. 63103-USA), at exemplary concentrations from 2.0 uM, 2.5 uM, 5 uM, 10 uM, up to 20 uM, for one day. Nonlimiting examples of mTeSR Media include, cGMP mTeSR™1, mTeSR™1, TeSR™2, TeSR™-E7™, TeSR™-E5, TeSR™-E6, ReproTeSR™, mTeSR™3D, etc., defined, serum-free media for culture of human ES, iPS, pluripotent stem cells, and the like). Clean iPSCs cells daily by removing differentiated cells to maintain a spontaneous differentiation free culture for optimal skeletal muscle differentiation. In one embodiment, 3 wells of a 96 well plate containing iPSCs, maintained at 70-80% confluence is suggested for use to start differentiation.

More specifically, Stage 1 skeletal muscle induction: Step 1. Dissociate iPSCs with Accutase (e.g. of a cell detachment solution) for 5 min.; Step 2. Resuspend cells in phosphate buffered saline (PBS) in a 15 mL conical tube; Step 3. Centrifuge the cells for 5 min (minutes) at 1000 RPM (revolutions per minute) for spinning cells gently to the bottom of the tube; Step 4. Aspirate media without disturbing the cell pellet in the bottom of the tube, then resuspend cells in skeletal muscle induction media 1, DMEM/F12, (see, Table 3); Step 5. Count the number of live cells (in part by exclusion staining the dead cells), e.g. using an automated cell counter: Take out 10 ul of cell suspension from the tube, mix with 10 ul of dye (1:1), e.g. in Trypan blue dye for staining dead cells, mix well, load mixture in cell counter chamber to count; Determine live cell numbers per ml, then Step 6. Plate single cells with appropriate number of cells, as suggested herein, on a Matrigel coated plate in mTeSR Media supplemented with Rock Inhibitor (Y-27632), see exemplary materials and concentrations above, for one day; Step 7. On the next day, switch the Stage 1 media to DMEM/F12 (1:1) supplemented with exemplary concentrations of 3 uM CHIR99021, 0.5 uM LDN193189; Step 8. Change media everyday until day three; then Step On Day three, supplement the existing media with an exemplary concentration of 20 ng/mL bFGF and continue feeding for additional seven days. Media should be change on a daily basis.

TABLE 3
Skeletal Muscle Induction Media 1.

| | Exemplary | Exemplary | Exemplary |
|---|---|---|---|
| Stage 1 Media | Concen- | Catalog | Company |
| Components | tration | number | (Source) |
| DMEMSF/12 | 1:1 | NA (not | Sigma-Aldrich, St. |
| (Dulbecco's | | available) | Louis, MO 63103- |
| Modified | | | USA |
| Eagle | | | |
| Medium/ | | | |
| Nutrient | | | |
| Mixture | | | |
| F-12 Ham) | | | |
| CH1R99021 | 3 uM | M60002 | Xcess |
| | | | Biosciences, |
| | | | Inc. (XcessBio)., |
| | | | San Diego, CA |
| | | | 92130, USA |
| LDN193189 | 0.5 uM | S2618 | Selleck |
| | | | Chemicals, |
| | | | Houston, TX |
| | | | 77054, USA |
| bFGF (Basic | 20 ng/mL | NA | Sigma-Aldrich, St. |
| fibroblast | | | Louis, MO 63103- |
| growth factor) | | | USA; PeproTech, |
| | | | Rocky Hill, NJ |
| | | | 08553-USA |

Stage 2—Commitment to Myoblasts. 1. After 10 days of incubation (e.g. 7 days incubation in complete skeletal muscle induction media 1), the media is changed to a DMEM/F12 (1:1) supplemented with exemplary concentrations of 10 ng/ml HGF, 2 ng/ml IGF and 0.5 uM LDN193189 (Skeletal Muscle Induction Media 2) for two days of incubation, see Table 4; If cells are too confluent by day 12-14, cells should be dissociated and replated on ECM, e.g. Matrigel coated surfaces at recommended cell densities, mentioned above, for optimal results; and 2. On day 12, cells were cultured with DMEM/F12 (1:1), with exemplary concentrations of 15% KSOR supplemented with with an exemplary concentrations of 2 ng/ml IGF (incomplete Skeletal Muscle Induction Media) for up to four days.

TABLE 4
Skeletal Muscle Induction Media 2.

| Stage 2 Media | Exemplary | Exemplary | Exemplary |
|---|---|---|---|
| Components | Concentration | Catalog number | Company (Source) |
| DMEMS/F12 | (1:1) | NA (not | Sigma-Aldrich, |
| | | available) | St. Louis, |
| | | | MO 63103-USA |
| CH1R99021 | 3 uM | M60002 | Xcess Biosciences, |
| | | | lee, (XcessBio), |
| | | | San Diego, |
| | | | CA 92130, USA |
| LDN193189 | 0.5 uM | S2618 | Selleck Chemicals, |
| | | | Houston, |
| | | | TX 77054, USA |
| bFGF | 20 ng/mL | NA | Sigma-Aldrich, |
| | (at least 1, 5, | | St. Louis, MO |
| | 10, 20, 30, up | | 63103-USA |
| | to 50 ng/ml) | | |
| HGF (hepatocyte | 10 ng/mL | NA | R&D Systems, |
| growth factor) | at least 2, 4, | | Minneapolis, |
| | 5, 10, 20, 30, | | Minnesota, USA |
| | 60, 100 up to | | |
| | 200 ng/ml) | | |
| IGF (insulin-like | 2 ng/mL | NA | PeproTech, |
| growth factor) | (at least 1.5, | | Rocky Hill, |
| | 2, 5, 10, 60, | | NJ 08553-USA |
| | 100 up to | | |
| | 200 ng/ml) | | |

Stage 3 Maturation: For differentiation of myoblasts into myotubes and for maintenance of Skeletal muscles: 1. On Day 12, 13 or 14, media was changed to DMEM/F12 (1:1), with exemplary concentrations of 15% KSOR supplemented with 10 ng/mL HGF and 10 ng/mL IGF-1 (complete Skeletal Muscle Induction Media 3); 2. Change Media every other Day until used, up to day 40; and 3. Optional: Fix cell samples, up to day 40 (or day used), e.g. of fixative, 4% PFA (Paraformaldehyde) to stain for skeletal muscle markers, e.g. as described herein. Other fixatives may be used for immunostaining.

TABLE 5
Skeletal Muscle induction Media 3.

| | | Exemplary | Exemplary |
|---|---|---|---|
| Stage 3 Media | Exemplary | Catalog | Company |
| Components | Concentration | number | (Source) |
| DMEM/F12 | (1:1) | NA (not | Sigma-Aldrich, |
| | | available) | St. Louis, MO |
| | | | 63103-USA |
| KOSR (Knockout | 15% | KnockOut ™ | Gibco ™ |
| Serum Replacement) | | SR | KnockOut ™ |
| | | 10828028 | Serum |
| | | | Replacement |
| HGF | 10 ng/mL | NA | R&D Systems, |
| | (at least 2, 4, | | Minneapolis, |
| | 5, 10, 20, 30, | | Minnesota, USA |
| | 60, 100 up to | | |
| | 200 ng/ml) | | |
| IGF-1 (insulin-like | 10 ng/mL | NA | PeproTech, |
| growth factor 1) | (at least 1.5, | | Rocky Hill, NJ |
| | 2, 5, 10, 60, | | 08553-USA |
| | 100 up to | | |
| | 200 ng/ml) | | |

The exemplary protocol described here for differentiating hSkMCs was used on ECM coated substrates, such as plates and microfluidic channels. For examples of ECM, plates and channels were coated with Matrigel, while microfluidic channels were coated with Laminin (non-cross-linked) and cross-Linked Laminin, as described herein. Seeding densities for the chips were used as described for the experiments, where either ihSkMCs were differentiated as described here, as one example, starting myotube differentiation on D1 in Stage 1 Skeletal Muscle Induction Media (incomplete).

B. Extracellular Matrix (ECM) Cross-Linking Effects on Myotube Structure and Stability in Chips.

As one embodiment, a single channel chip (e.g. Quad chip: as a 4 single channel chip) was used initially for determining stages of muscle cell maturation on a chip and numbers of seeded cells that provide viable cultures in relation to chips coated with ECM.

In some embodiments, an extracellular matrix (ECM) layer is provided to coat (cover) the entire surface of the lower channel (bottom, sides and top) for growing human skeletal striated muscle cells. In one embodiment, Laminin was used as an exemplary ECM component for coating the surface. In another embodiment, a cross-linker chemical was used for cross-linking Laminin molecules. As an exemplary cross-linker chemical, Sulfo-SANPAH was used.

Experiment 2: showed that Sulfo-SANPAH cross linked ECM provides more stability to hSkMCs. Sulfo-SANPAH cross-linked ECM enables formation of almost 2-fold more MHC positive multinucleated fibers. Further, more nuclei per myo-tubes with cross-linked ECM. In fact, a 3-fold higher number of nuclei in MHC myo-fibers seeded on Sulfo-SANPAH cross-linked ECM-Laminin was observed over Laminin alone.

1. Human Skeletal Muscle Cells: Extracellular Matrix.

a. Extracellular Matrix (ECM).

In some embodiments, an extracellular matrix (ECM) layer is provided to coat (cover) the entire surface (bottom, sides and top) of the lower channel for growing human skeletal striated muscle cells. In one embodiment, laminin was used as an exemplary ECM component for coating the surface. In another embodiment, a cross-linker chemical was used for cross-linking laminin molecules. As an exemplary cross-linker chemical, Sulfo-SANPAH was used.

Sulfo-SANPAH cross-linked ECM enables formation of almost 2-fold more MHC positive multinucleated fibers. Further, more nuclei per myo-tubes with cross-linked ECM. In fact, a 3-fold higher number of nuclei in MHC myo-fibers seeded on Sulfo-SANPAH cross-linked ECM-laminin was observed over laminin alone.

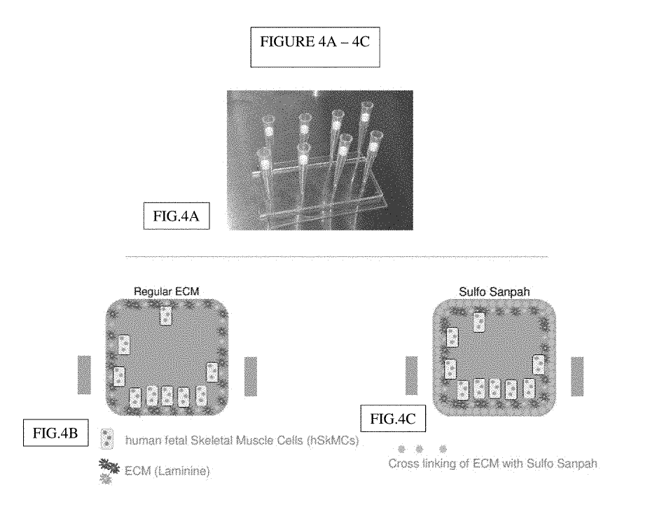

FIG. 4A-4C: Shows one embodiment of a human skeletal muscle cell culture hSkMC-In-Chip: Extracellular Matrix (ECM) use for hSkMCs-In-Chip. In one embodiment, the chip is a Quad chip.

FIG. 4A: shows a picture of a single channel (Quad) Chip with pipette tips used to block channels for coating the inside surfaces with an ECM layer then seeded with human skeletal muscle cells (hSKMCs).

FIG. 4B: shows a schematic illustration of a cross-sectional view of the quad channel with ECM as Laminine (purple and blue stars) with hSkMCs as yellow-spotted blocks.

FIG. 4C: shows a schematic illustration of a cross-sectional view of the quad channel with ECM as Laminine (purple and blue stars) with hSkMCs as yellow-spotted blocks and a representative cross linking of ECM as yellow stars, e.g. with Sulfo-SANPAH.

b. Extracellular Matrix (ECM) Cross-Linking Effects on Myotube Structure and Stability in Chips.

This example shows one embodiment of a set up and time course for culturing Human Muscle Cells in-Chip: providing non-contracting myotubes on ECM coated chips. As one embodiment, a single channel chip (e.g. Quad chip: as a 4 single channel chip) was used initially for determining stages of muscle cell maturation on a chip, effects of ECM, and numbers of seeded cells that provide viable cultures in relation to chips coated with ECM. In this embodiment, muscle cells grown without nerve cells present did not show spontaneous contractions of myotubes.

Experiment 1 showed that hSkMC seeding density at 3×10⁶ cells/ml, but loss of cells 24 h after contracting activity

As one example, Sulfo-SANPAH cross-linked ECM enables formation of almost 2-fold more MHC positive multinucleated fibers. Further, more nuclei per myo-tubes with cross-linked ECM. In fact, a 3-fold higher number of nuclei in MHC myo-fibers seeded on Sulfo-SANPAH cross-linked ECM-Laminin was observed over a Laminin coating without the use of a cross-linker.

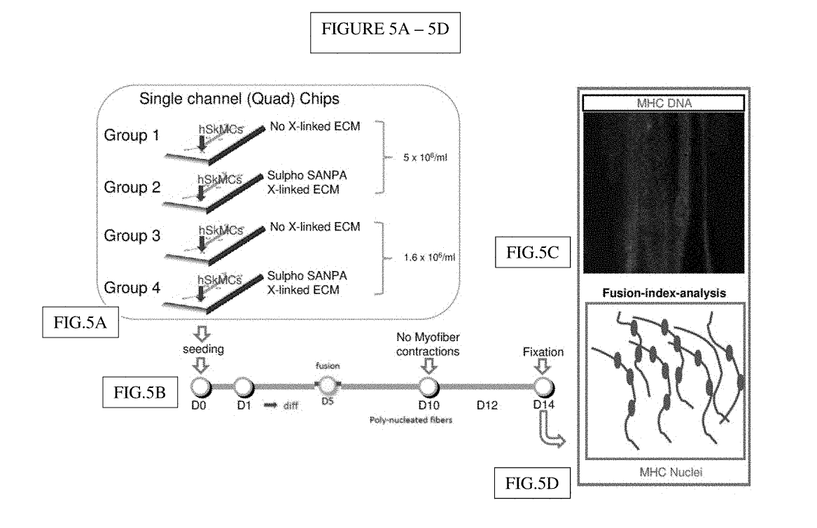

FIG. 5A-5D: shows one embodiment of a human muscle cell culture in-chip: Set Up and Time Course for producing multinucleated myofibers that are not contracting.

FIG. 5A: Single channels of Quad Chips were seeded with human skeletal muscle cells (hSKMCs), Group 1 and Group 2: 5×10⁶/ml cells; Group 3 and Group 4: 1.6×10⁶/ml cells. Groups 1 and 3 do not have cross (X)-linked ECM while Groups 2 and 4 have exemplary Sulpho SANPA X-linked ECM.

FIG. 5B: shows a schematic experimental timeline: Seeding cells on Day (D) 0. D1: Inducing differentiation. D5 observing fusion of myoblast cells. D10: Screening for myo-fiber contraction in cultures that were not stained for analysis; observing polynucleated fibers but no myofiber contractions. D14 Fixing cells and fusion-index-analysis.

FIG. 5C: Day 14: Fixation and fusion-index-analysis based upon staining for myosin heavy chain (MHC) (red) and nuclei (DNA) (shown in blue).

FIG. 5D: Shows a schematic illustration of multinucleated myofibers in MHC (red) and nuclei (DNA) (blue).

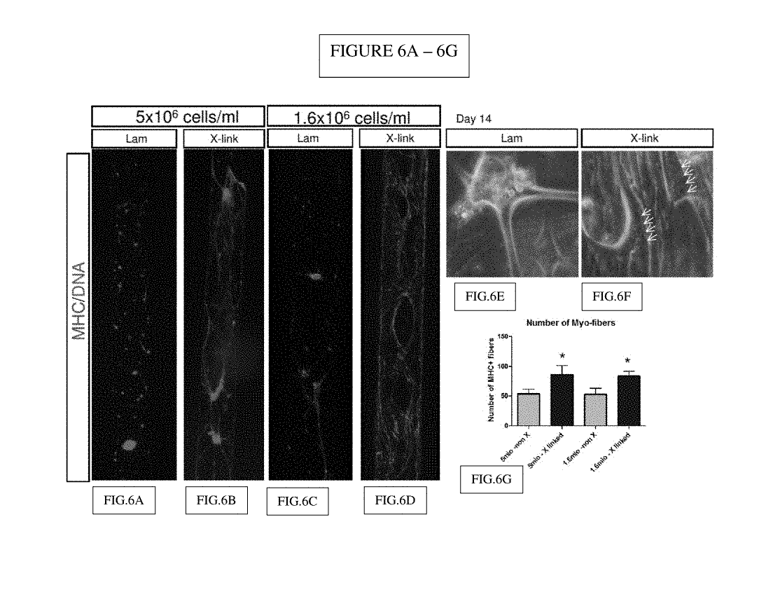

FIG. 6A-6G: shows Human Skeletal Myoblast-Derived Poly-Nucleated Fibers growing in microfluidic chips where Sulfo-SANPAH cross-linked ECM enables formation of almost 2-fold more MHC positive multinucleated fibers.

FIG. 6A-6D: show fluorescent micrographs of immunostained myosin heavy chain (MHC) (red) myo-fibers and DAPI stained nuclei (DNA) (shown in blue) comparing cultures started at the 2 different densities (FIGS. 6A-B: 5×10⁶/ml cells and FIGS. 6C-6D: 1.6×10⁶/ml cells) with and without cross-lined (X-link) ECM-Laminin (Lam).

FIGS. 6E-6F: show phase contrast micrographs of Day 14 cells grown on Laminin (Lam) and cross-linked (X Link) ECM-Laminin (Lam), respectively. More MHC positive multinucleated fibers are observed with X-Linked Laminin after 14 days. White arrows point to 2 exemplary multinucleated myotubes

FIG. 6G: shows a graph comparing number MHC+ myo-fibers to the treatments shown in FIGS. 6A-6D where at both cell densities the number of myofibers growing on x-Linked ECM is almost 2-fold more than fibers grown on regular, non-cross-linked, ECM.

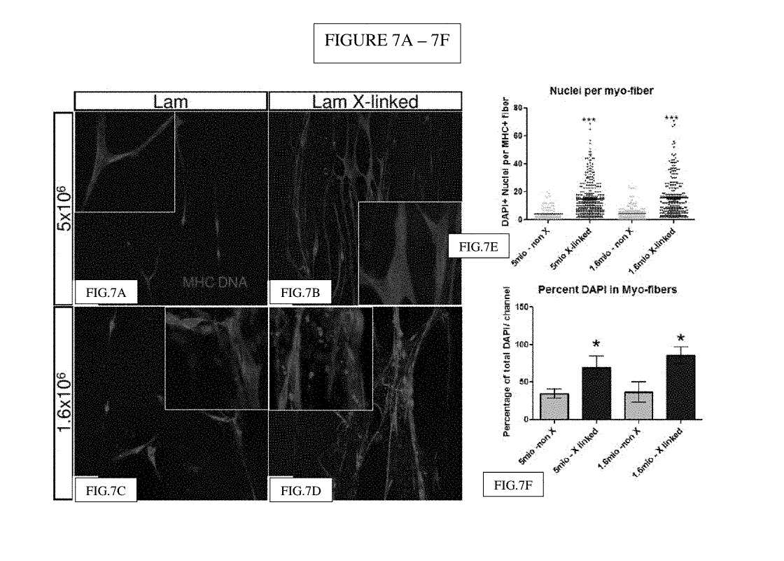

FIG. 7A-7F: shows Human Skeletal Myoblast-Derived Poly-Nucleated Fibers growing in microfluidic chips comparing non-cross-linked to cross-linked ECM (Laminin) where more nuclei per myo-tubes are observed growing on cross-linked ECM.

FIG. 7A-7D: show fluorescent micrographs of immunostained myosin heavy chain (MHC) (red) myo-fibers and DAPI stained nuclei (DNA) (shown in blue) comparing cultures started at the 2 different densities with inserts showing higher magnifications of presumptive myo-fibers for each treatment.

FIGS. 7A-7B: 5×10⁶/ml cells and FIGS. 6C-D: 1.6×10⁶/ml cells) with Laminin (Lam) and with cross-linked (X-linked) Laminin-ECM.

FIGS. 7E-7F: Show a 3-fold higher number of nuclei in MHC myo-fibers seeded on exemplary Sulfo-SANPAH cross-linked ECM by graphical comparisons.

FIG. 7E: shows a graph comparing DAPI+ nuclei per MHC+ fiber for determining myo-fiber at the 4 treatments shown.

FIG. 7F: shows a graph comparing percentage of total DAPI+ per channel, i.e. percentage of DAPI in myo-fibers at the 4 treatments shown in FIG. 7A-D.

IV. Combining MN-On-Chip With hSkMC-On-Chip for Providing Embodiments of a NMJ-on-Chip.

In one embodiment, the starting material for generating at least one cellular component for the NMJ generated on a microfluidic device (or simply “NMJ-on-chip”) comprises stem cells (e.g. see the protocols in Examples, and below). In particular embodiments, these stem cells may include, for example, induced pluripotent stem cells (iPS cells) or embryonic stem cells. In one embodiment, progenitor cells (derived from stem cells) related to neural lineages or cells directly reprogrammed into motor neurons, neural lineage progenitors, and the like, are employed/seeded on the chip. In one embodiment, progenitor cells (derived from stem cells) related to skeletal muscle lineages or cells directly reprogrammed into skeletal muscle cells, skeletal muscle multinucleated myotubes, skeletal muscle lineage progenitors, and the like, are employed/seeded on the chip. It is important to note that not all cell types involved in the NMJ-on-chip must be generated from stem cells. For example, the NMJ-on-chip may employ primary skeletal muscle cells. Techniques are known in the art to reprogam, expand and characterize human iPS cells from human skin or blood tissues of healthy subjects and diseased patients. For example, a non-integrating system based on the oriP/EBNA1 (Epstein-Barr nuclear antigen-1) episomal plasmid vector system can be used to avoid potential deleterious effects of random insertion of proviral sequences into the genome. See Okita K, et al., “A more efficient method to generate integration-free human iPS cells,” Nat Methods. 2011 May; 8:409. It is preferred that the iPSC lines so generated express the pluripotency markers (SSEA4, TRA-1-81, OCT3/4, SOX2) along with a normal karyotype. In the present invention, iPS cells are used to generate components of the NMJ-on-chip, e.g. neurons, etc. While in many cases, the iPS cells are from normal subjects, it is also contemplated that the iPS cells can be derived from patients exhibiting symptoms of disease. In one embodiment, the NMJ-on-chip is populated with cells derived from iPS cells from a patient diagnosed with a disorder of the nervous system, including but not limited to iPSC-derived motor neurons from Amyotrophic lateral sclerosis (ALS) patients. See D. Sareen et al., “Targeting RNA foci in iPSC-derived motor neurons from ALS patients with C9ORF72 repeat expansion” Sci Transl Med. 2013 Oct. 23; 5(208): 208ra149.

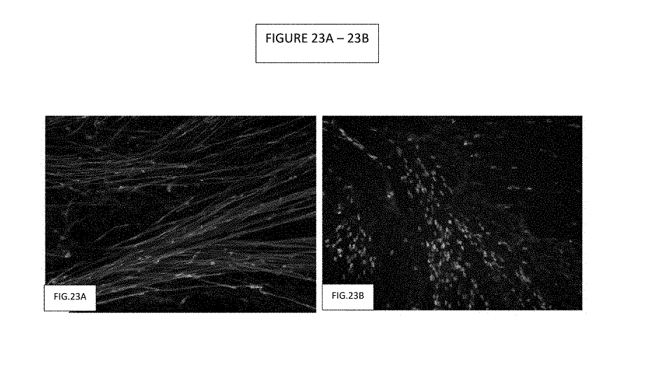

As one example, FIG. 23A-23B: shows exemplary fluorescent micrographs of NMJ-On-Chips using iPSC derived Myo-fibers (iSKMCs) as superimposed (co-localized images) of neurons and myotubes. (Experiment 5).

FIG. 23A: shows a fluorescent micrograph of nerve axons (red) parallel to multinucleated (blue) muscle heavy chains within muscle myofibers (green) showing separation between internal myosin and external nerve fibers. Myosin (MHC: myosin heavy chain) (green), neuronal nerve fibers TuJ1 (red) and DNA (DAPI) (shown in blue)

FIG. 23B: shows a fluorescent micrograph view on end (as compared to the orientation in FIG. 23A) for a different view, i.e. x-z image, of muscle Myogenin (green), nerve TuJ1 (red) and DNA (DAPI) (shown in blue) where nuclei superimposed on the muscle staining shows light blue, see example at the white arrow.

In one embodiment, the present invention contemplates differentiating “stem-cell derived cells” on the chip, i.e. in a microfluidic environment. The term “stem-cell derived cells” refers to cells derived from stem cells that fall on a spectrum of differentiation. For example, in one embodiment, induced motor neuron progenitor cells (including but not limited to, iPSC-derived spinal neural progenitors) are derived from induced pluripotent stem cells, but they are not fully differentiated. In one embodiment, induced motor neuron progenitor cells are differentiated on-chip to generate motor neurons, and ultimately mature motor neurons. Thus, in one embodiment, the present invention contemplates a method of culturing cells, comprising: a) providing a microfluidic device (optionally comprising a membrane, said membrane comprising a top surface and a bottom surface); b) seeding induced motor neuron progenitor cells (optionally on said top surface and optionally skeletal muscle cells on said bottom surface so as to create seeded cells); c) exposing said seeded cells to a flow of culture media for a period of time (days to weeks to months) under conditions such that said at least a portion of said progenitor cells differentiate into motor neurons (and preferably wherein said motor neurons display a mature phenotype based on testing described herein or staining). Further, at least a portion of said progenitor cells differentiate into skeletal muscle cells (and preferably wherein said skeletal muscle cells display a mature phenotype based on testing described herein or staining). In a preferred embodiment, at least a portion of the skeletal muscle cells form multinucleated myotubes. In yet another embodiment, at least a portion of the multinucleated myotubes are striated. In one embodiment, the method (optionally) further comprises e) culturing said seeded cells under conditions such that said skeletal muscle cells on said bottom surface form neural muscular junctions.

In some embodiments of a NMJ-on-a-chip, neural cell cultures were seeded into chips following the seeding of hSMCs, described above, either on the same day, 18 hours later, the following day, or up to 9 days after hSMCs had been seeded onto the chip. The chips were cultured for 14 days and fixed and stained for relevant markers. In some embodiments, confocal microscope imaging shows proximity of cells in a z-stack image.

Thus in some embodiments, neural cells in the top channel of the microfluidic device and hSMCs on the bottom channel of the microfluidic device are shown in close proximity.

The attached cells were then tested for markers to confirm their identity, e.g. ICC. ICC overlay data: By overlaying images taken after staining the cells, specific cell identification can be combined with original activity traces (e.g. calcium flux images, etc) to determine specific activities of individual cell types in the chip.

In some figures shown herein, images from a microfluidic chip wherein at least a portion of a MN (i.e. the terminal bulb) has transmigrated the membrane and contacted the hSMCs on the other side. In some examples, MN are shown in red against the green stained hSMCs.

Thus in one embodiment a vertical 2D projection of a 3D confocal stack of images slices is imaged, which allows for visualization of the neurons and hSMCs together, even though they are not in the same imaginary plane on the microfluidic device. hSMCs display a MHC marker, while the neurons are positive for TUJ1, for example. DAPI (4′,6-diamidino-2-phenylindole) is used as a fluorescent stain for DNA (deoxyribonucleic acid) in nuclei.

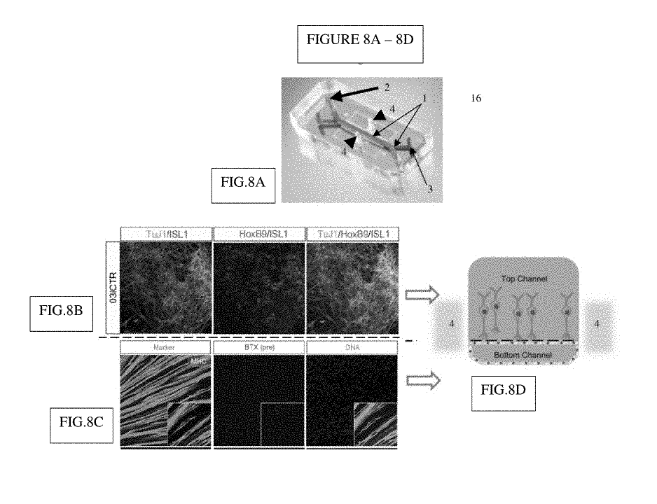

As one example, FIG. 8A-8D: shows one embodiment of a Human iPS-Derived MN and Muscle Cell Co-Culture in-a Tall Channel Microfluidic Chip.

FIG. 8A: shows a picture of a tall channel microfluidic chip (16) in one embodiment seeded with MNs at day 12 of culture into the port (2) of the upper (blue) channel (thick arrow) (1) and human skeletal muscle cells into the port (3) of the lower (red) channel (1) at the end of the other channel (thin arrow). The arrowhead points to a vacuum chamber (4), for optional use.

FIG. 8B: shows iPSC-derived MNs seeded into the upper channel forming a neural network stained with TUJ1 (green); Islet1 (ISL1) (blue); indicating early motor neurons, and Islet1 (ISL1) (blue); HoxB9 (red); indicating more mature motor neurons, while the third frame is a superimposed image showing both early and more mature motor neurons.

FIG. 8C: shows skeletal muscle cells seeded into the lower channel stained with myosin heavy chain (MHC) (green) with an insert showing myofibers at a higher magnification; α-bungarotoxin BTX (pre-BTX) (red), for identifying AchR in the motor end plate, with an insert showing stained cells at a higher magnification; and DNA in nuclei stained then fluoresced in the blue range, with an insert showing myofibers (green) at a higher magnification with unstained regions that likely correspond to multinuclear areas in the myofibers; and

FIG. 8D: shows a schematic illustration of a vertical cross section of a tall channel microfluidic chip where MNs from a Day 12 culture seeded onto the chip develop cell bodies containing nuclei (purple circles), axons and terminal areas next to the membrane separating the top from the bottom channel containing human skeletal muscle cells growing around the edge of the channel.

### V. Medium Optimization to Reduce Spontaneous Contraction Rates in NMJ-On-Chip for Providing a Functional NMJ-On-Chip.

By day 10 of cultures, observations of myotubes showed high rates of spontaneous contractions. In fact, a loss of myotubes starting around 24 hours was observed after start of spontaneous contractions. Therefore, experiments were designed for identifying media that would reduce spontaneous contractions in cultures. It was determined that spontaneous contraction rates of muscle cells should be lowered in order to determine whether spontaneous contractions were effecting longer term viability, and for use in testing potential treatments, including agents, for increasing contraction rates. Therefore, the following embodiments are provided for developing medium for lowering spontaneous contraction rates. Media was tested that included at least one agent for reducing spontaneous myotube contraction rates. In part, rates were artificially reduced in order to allow testing of agents for altering muscle contractions, e.g. increasing muscle contraction rates.

Thus, in some embodiments, a media for lowering contraction rates was developed, e.g. CoM media was developed and used for perfusing NMJ-on-chips. As used herein, “COM” or “coM” or “CoM” or “co-media” refers to a culture media as formulated in Table 1, Day 12-xx (see above), which in addition to Iscove's Modified Dulbecco's Media/Ham's F-12 Nutrient Mixture (IMDM/F12), Non-Essential Amino Acids (NEAA), B27 supplement (B27), e.g. Gibco™ B-27 Serum Free Supplement (plus vitamin A), N-2 Supplement (N2), e.g. Gibco™, PSA, Compound E and DAPT, e.g. STEMCELL Technologies Inc., Cambridge, Mass. 02142-USA, all-trans RA, e.g. STEMCELL Technologies Inc., purmorphamine (or SAG), both available, e.g. STEMCELL Technologies Inc., Cambridge, Mass. 02142-USA, db-cAMP, Ascorbic Acid, e.g. STEMCELL Technologies Inc., Cambridge, Mass. 02142-USA, Glial cell-derived neurotrophic factor (GDNF), Promega Corporation, Brain-derived neurotrophic factor (BDNF), e.g. (Sigma-Aldrich), and VPA (valproic acid), e.g. (Sigma-Aldrich), includes 2% FBS serum, as one example of a media for reducing spontaneous skeletal muscle contractions in co-cultures of MNs and hSkMCs. Media components are listed with an example of an exemplary source.

In this example, exemplary embodiments are provided for a Human iPS-Derived MN and Muscle Cell Co-Culture in-Chip for use in testing for variable effecting longer term viability of cells and for using chips in testing pharmacology agents, i.e. for use in treating NMJ related diseases.

Experiment 1: Human iPS-Derived MN and Muscle Cell Co-Culture in-Chip.

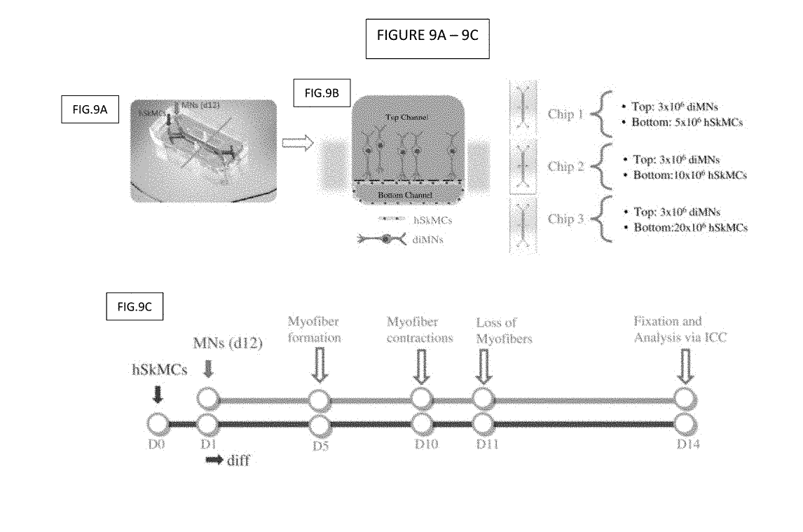

Day 0: seeding hSkMCs; Day 1: (18 h later) seeded diMNs (d12); Day 5: observation of formation of myotubes; Day 10: observation of myofiber contraction; Day 11: observation of progressive loss of myofibers; Day 14: fixation and analysis. There was a continuous loss of myo-tubes after day 11-24 hours, after last observation of spontaneous myo-tube contractions. Further, the use of flow during culture increases loss of myo-tubes. See, FIG. 9 for a schematic illustration and numbers of cells in the different replicates for comparing effects of initial seeding densities.

Experiment 1 showed that hSkMC seeding density at 3×10⁶ cells/ml, but loss of cells 24 h after contracting activity.

FIG. 9A-9C: Shows one embodiment of a Human iPS-Derived MN and Muscle Cell Co-Culture in-a microfluidic Chip.

FIG. 9A is a picture of an exemplary microfluidic chip where day 12 MNs are seeded into the top (upper-blue) channel and hSkMCs are in the bottom (lower-red) channel;

FIG. 9B shows a schematic illustration of an exemplary cross section of NMJ microfluidic chip with day 12 MNs in the top channel and hSkMCs in the bottom channel with 3 sets of Experimental Chips for comparing cell densities at the time of seeding: Chip 1: top: 3×10⁶/ml diMN cells and bottom: 5×10⁶/ml hSkMC cells; Chip 2: top: 3×10⁶/ml diMN cells and bottom: 10×10⁶/ml hSkMC cells; and Chip 3: top: 3×10⁶/ml diMN cells and bottom: 20×10⁶/ml hSkMC cells.

FIG. 9C: shows a schematic illustration of a timeline showing co-culture of hSkMCs seeded Day (D) 0 with differentiation (diff) initiated on D1, Day 12 MNs seeded D1, Myofiber formation on D5, myofiber contractions observed D10, a loss of myofibers observed on D11, with fixation and analysis by ICC on D14.

A. Experimental System for Testing Media to Reduce Spontaneous Muscle Contraction Rates.

The following experiments were designed for identifying media components that would lower spontaneous contraction rates.

### Experiment 3: Testing Media Components for Reducing Spontaneous Muscle Contractions.

Top: 3×10⁶ diMNs and Bottom: 20×10⁶ hSkMCs, as tested in 3 different groups of either cells seeded on top, bottom or both, in media harvested from diMNs/hSkMCs cultures or coM.

Experiment 3 showed improved hSkMCs in-chip integrity. However this was lost 48 h after contraction activity occurred in diMN/hSkMC media.

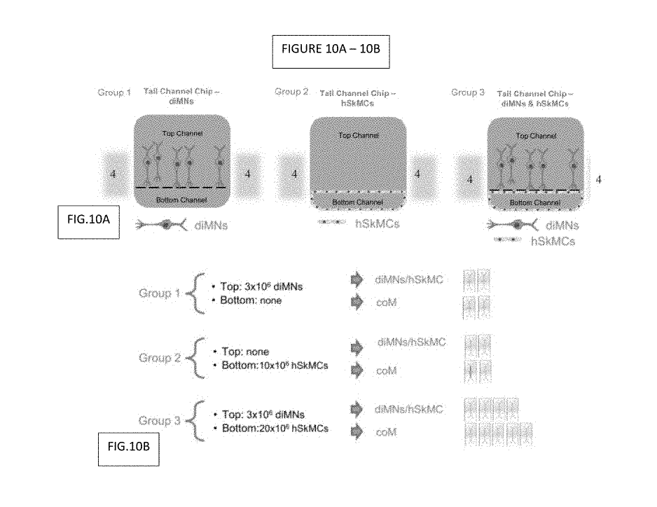

FIG. 10: shows one embodiment of an experimental system (Experiment 1) as a schematic illustration for testing medium to reduce spontaneous contractions of cells in the microfluidic tall channel chip. Experimental Groups 1-3 directly compare medium harvested from diMNs/hSkMC cultures with coM media in chips containing induced motor neurons (diMNs: Motor-neuron-on Chip) and human Skeletal Muscle Cells (hSkMCs-on-Chip), each cell type growing alone on chips then combined in the same chip in the same media (upper and lower channel) for providing a neuronal-muscular-junction (NMJ-on-Chip).

FIG. 10A: Group 1: shows a schematic illustration of the tall channel chip, with vacuum chambers (4), diMNs in the top channel but no cells in the bottom channel. Group 2: shows a schematic illustration of the tall channel chip with no cells in the top channel but with hSkMCs in the bottom channel. Group 3: shows a schematic illustration of the tall channel chip with diMNs in the top channel and hSkMCs in the bottom channel for providing a NMJ-on-Chip.

FIG. 10A: shows a schematic illustration of cells numbers and media used for growing cells: Group 1: Top: 3×10⁶ diMNs Bottom: none. Group 2: Top: none. Bottom: 10×10⁶ hSkMCs. Group 3: Top: 3×10⁶ diMNs. Bottom: 20×10⁶ hSkMCs.

B. Reducing Spontaneous Myotube Contractions at Day 10 (D10).

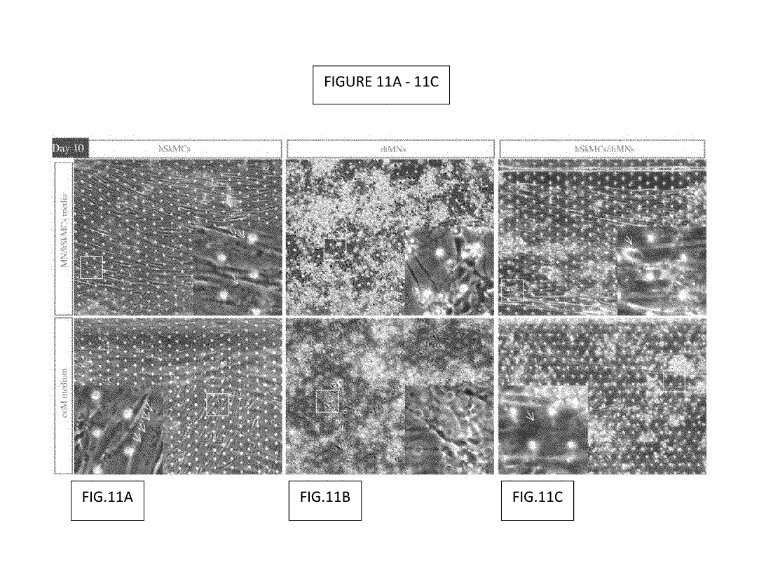

By day 10 of cultures, myotubes showed high rates of spontaneous contractions, see, FIG. 11A-11C. Therefore, experiments were designed for identifying media that would reduce spontaneous contractions in cultures.

FIG. 11A-11C: Shows human skeletal muscle cells (hSkMCs) forming myofibers within 8 days post seeding (co-cultures) having spontaneous myo-tube contractions at Day (D) 10 culture that are reduced by using conM culture medium in a microfluidic chip.

FIG. 11A: shows micrographs of hSkMCs growing in chips. White arrows in the magnified region point to multinucleated muscle cell fibers, of which there appears to be more nuclei per fiber in the coM medium;

FIG. 11B: shows micrographs of diMNs growing in chips; and

FIG. 11C: shows micrographs of shSkMCs/diMNs grown in MN/hSkMCs media (upper row of micrographs) and coM medium (lower row of micrographs) growing in chips. Spontaneous myo-tube contraction was observed only in diMNs/hSkMC co-cultures. White arrows in the magnified region point to contacts of MN with a muscle cell fiber.

Inserts show higher magnified areas of cells outlined in the white box for each micrograph.

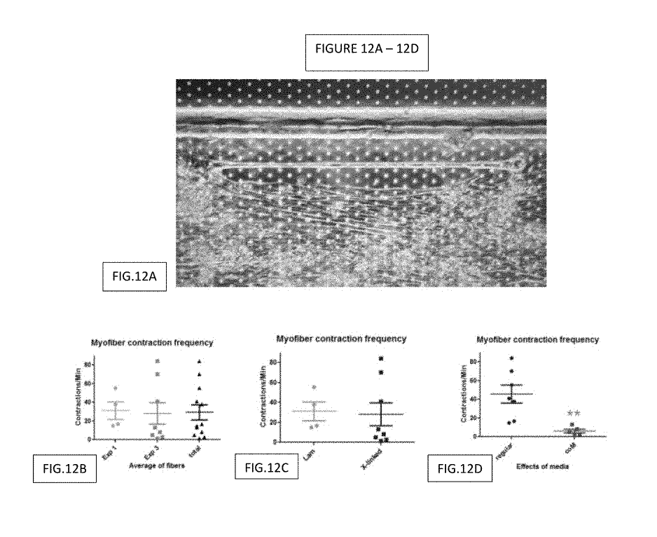

FIG. 12A-12D: Shows human skeletal muscle cells (hSkMCs) as myofibers with spontaneous myotube contraction at Day (D) 10 (Experiment 3).

FIG. 12A: shows a micrograph of hSkMCs as myotubes growing on top of a membrane of the microfluidic chip in coM media.

FIG. 12B: shows a graph comparing contractions per minute for a myofiber contraction frequency with an average of fibers in two experiments (Experiment 1 and 3) that were combined for a total estimation of myofiber contraction frequency.

FIG. 12C: shows a graph comparing contractions per minute for myofibers having an increased myofiber contraction frequency between Laminin vs. cross linked Laminin ECM, at about the same frequency as shown in FIG. 12B.

FIG. 12D: shows a graph comparing contractions per minute for myofibers grown in regular media compared to a culture grown in coM media. When cultured in coM, contraction frequency is around 25% less compared to regular medium conditions.

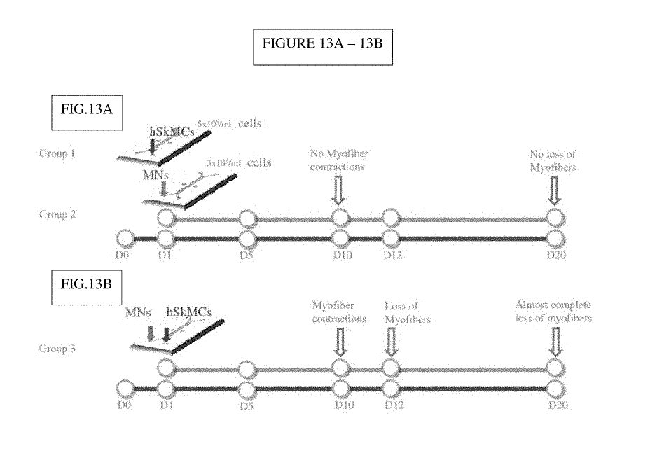

FIG. 13A-13B: Shows schematic illustrations of experimental timelines for comparing co-cultures of hSkMCs with MNs, with and without coM media. The use of coM media allows the control of myofiber structure and function over time.

FIG. 13A: shows a schematic illustration of a timeline and cell densities for Group 1 and Group 2 in coM: hSkMCs seeded at 5×10⁶/ml cells and MNs seeded at 3×10⁶/ml cells. hSkMCs seeded Day (D) 0 with differentiation (diff) initiated on D1, Day 12 MNs seeded D1 (as one example 18 h later), D5 formation of myotubes & medium switch to coM, no myofiber contractions observed D10, no loss of myofibers observed on D12, fixation and analysis by ICC on D14, duplicate chips on D20 showed no loss of myofibers.

FIG. 13B: Shows a schematic illustration of a timeline and cell densities for Group 3: hSkMCs seeded with MNs: Day 0: seeding hSkMCs; Day 1: (18 h later) seeded diMNs (d12); Day 5: formation of myotubes, no medium switch; Day 10: observation of myofiber contraction; Day 11: observing progressive loss of myofibers; Day 14: fixation and analysis by ICC; in chip cultures left to D20, there is almost a complete loss of myofibers.

VI. Co-Localization of iPS-Derived MNs and Muscle Cells Showing Potential Formation of NMJs in Microfluidic NMJ-On-Chip.

During the development of one embodiment of a functional NMJ-on-Chip, method steps for a successful motor neuron-on-chip are as follows: obtain patient iPSC-derived MNs, grown under conditions for inducing expression of certain neuronal markers by day 12, develop a successful skeletal muscle-on-chip: containing contractile tissue (i.e. myofibers), then co-culture skeletal muscle cells and neuronal cells on microfluidic chips under conditions to stop spontaneous contraction by adding blockers, such as calcium channel blockers, sodium channel blockers, tetrodotoxin (TTX), which is a potent blocker of voltage-gated calcium channels, and the like, to the media. Use immunohistochemistry (ICH) to identify characteristics of NMJs. Chip components include membranes with a pore Dia (diameter) of 7 μm spacing 40 μm Hex packed, Thickness: 50 μm, PDMS, Extracellular Matrix (ECM) provided is laminin (250 μg/ml).

Thus, the following embodiments are provided for identifying NMJs functional NMJ-on-chips, e.g., using co-localization of neuronal bulb markers, e.g. BTX, e.g. Tubb3 with muscle cells e.g. MHC.

### V. Using Microfluidic NMJ-On-Chip Under Flow for Longer Studies.

### Experiment 4: Extended Cultures up to Day 37.

Experiment 4 showed that hSkMC integrity in chip is expandable over time (in monoculture).

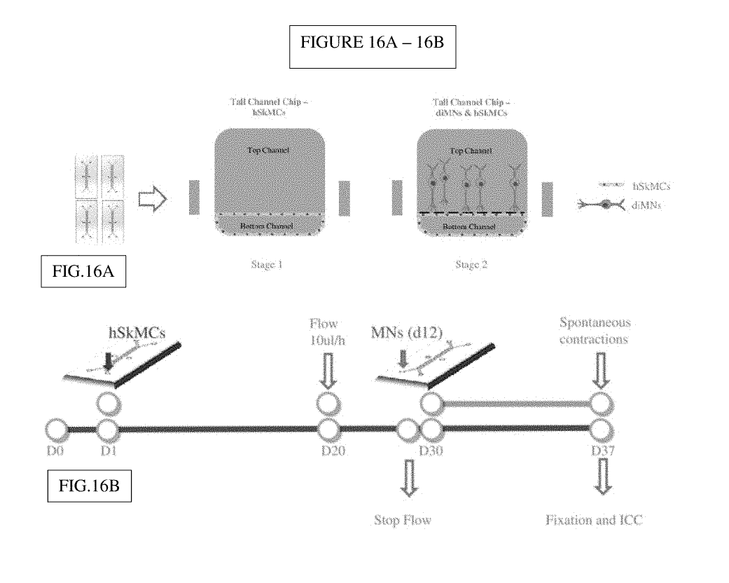

FIG. 16A-16B: shows schematic illustrations of tall channel microfluidic NMJ-on-chip with one embodiment of an experimental timeline (Experiment 4) set up and time course for comparing co-cultures of hSkMCs with MNs under flow.

FIG. 16A: shows a schematic illustration of a tall channel microfluidic chip, from left to right, view of vertical 2-channel chip (i.e. the top channel is above the bottom channel as shown in Stage 1, with hSkMCs covering the entire surface of the bottom channel, and Stage 2 with diMNs seeded into the top channel.

FIG. 16B: shows a schematic illustration of one embodiment of a timeline where hSkMCs are seeded Day (D) 0 with differentiation (diff) initiated on D1, D5: formation of myotubes & medium switch to coM media, then Day 7-10: no myofiber contraction, on Day 20 start muscle cells under flow at 10 ul/hour, continued to D29 when flow is stopped. Day 30: seed diMNs (d12) (not in coM media for observing baseline contractions). Day 37: myotubes are spontaneously contracting: fixation and analysis (including ICC).

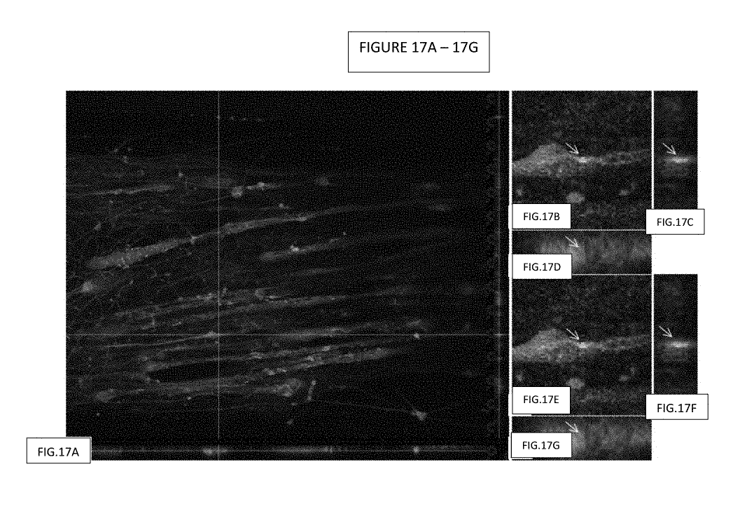

FIG. 17A-17G: shows an exemplary co-localization study of iPS-Derived MNs and Muscle Cells showing formation of NMJs between diMNs and hSkMCs (Experiment 4). Cells were stained with α-bungarotoxin (BTX) for identifying suggestive NMJ areas where motor end plate (green), neurons are stained with Tubulin beta-3 chain (Tubb3) (red) and muscle myosin heavy chain (MHC) (blue) were fluorescently imaged on individual channels then merged. The blue channel of MHC staining is not shown in FIG. 17A-17D.

FIG. 17A: shows a low power fluorescent micrograph where Tubb3 (red) neuronal staining shows neurite extension along myotubes with oval areas (green) suggestive of lower motor nerve termini whose distribution over a myotube suggests motor end plates.

FIG. 17B-G: shows higher power fluorescent micrographs of the suggestive NMJ areas (white arrows) are identified by superimposed staining i.e. co-localization, where the red stained nerve terminal neuron bulb is co-localized with BTX green staining of motor end plates producing a yellow NMJ.

FIG. 17E-17G: The blue channel of MHC staining is shown showing a MHC containing muscle fiber at the yellow stained NMJ.

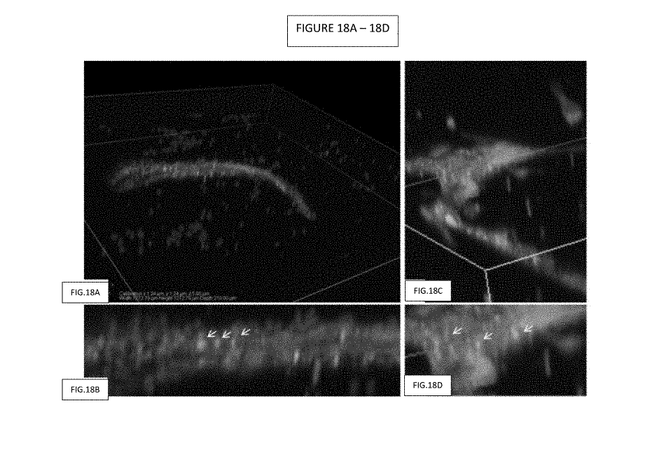

FIG. 18A-18D: shows fluorescent micrographs of stained cells in a microfluidic chip. Co-Localization Study of iPS-Derived MNs and Muscle Cells. Both diMNs and hSkMCs are in close proximity to each other as determined from initial ICC analysis and 3D reconstruction of confocal microscope images (i.e. combined z-stacks). A partial loss of myotubes were observed due to lack of ECM stability

FIG. 18A and FIG. 18B: α-bungarotoxin (BTX) for identifying the motor end plate (green), skeletal muscle marker, desmin, (red) and DNA (DAPI) (shown in blue). The red muscle fiber is multinucleated with numerous green motor end plates.

FIG. 18B: a higher magnification of FIG. 18A, 3 white arrows point to co-localization of α-bungarotoxin (BTX) for identifying the motor end plate (green) and skeletal muscle marker, desmin, (red) as olive, white dark orange areas depending upon concentration of stain.

FIG. 18C and FIG. 18D: motor end plate (green) BTX and neurofilament H non-phosphorylated (SMI 32) (red) and DNA (DAPI) (shown in blue).

FIG. 18D: a higher magnification of FIG. 18C, 3 white arrows point to co-localization of a motor end plate (green) BTX, neurofilament H non-phosphorylated (SMI 32) (red) as olive-white areas depending upon concentration of stain.

### VI. Using Microfluidic NMJ-On-Chip for Pharmacology Studies and Live Imaging of Cells Within Channels.

In this embodiment, an experimental time line (course) is described for seeding hSkMCs up to 9 days prior to seeding MNs in the upper channel. Spontaneous contractions are allowed to begin by removing CoM media at the start of the pharmacology assay.

Experiment 5 showed that pharmacology and imaging was possible for measuring functional NMJ interactions.

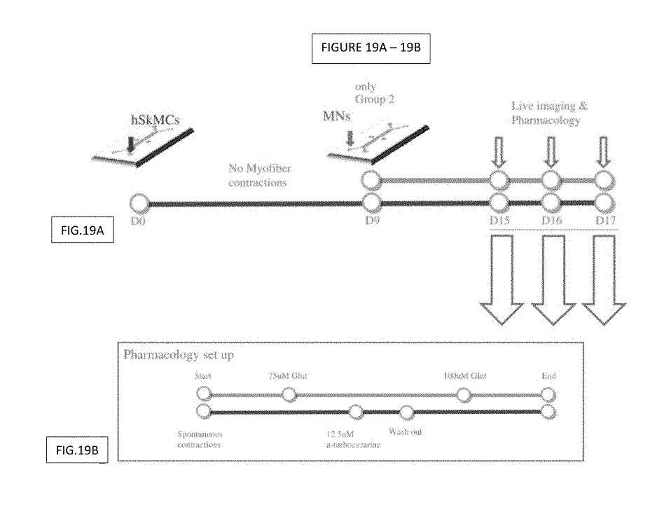

FIG. 19A-19B: shows schematic illustrations of one embodiment of experimental timelines for using NMJ-on-chips (Experiment 5) as a set up and time course for using co-cultures of hSkMCs with MNs for live imaging and pharmacology studies.

FIG. 19A: shows a schematic illustration of a tall channel microfluidic chip, seeded with hSkMCs at Day 0 (D0) in the bottom channel, culting up to D9, without observing muscle contractions, then D9 seeding diMNs (d12). In one embodiment only in Group 2. In some embodiments, more than one group of hSkMCs receive MNs. On days 15, 16 and/or 17, live imaging of pharmacology assays are done as shown schematically, for one example, in FIG. 19B.

FIG. 19B: shows a schematic illustration of one embodiment of a timeline where a NMJ-On-Chip with spontaneous contracting muscle fibers is used for a pharmacology study, i.e. testing agents for inducing or reducing muscle contractions on a baseline chip with or without spontaneously contracting myofibers, in one embodiment, treating NMJ chip with 75 uM Glutamine (Glut) in the NM (upper) channel), in one embodiment, treating NMJ chip with 12 uM alpha-turbocurarine in the hSkMC (lower) channel), in one embodiment, washing out alpha-turbocurarine, in one embodiment, treating NMJ chip with 100 uM Glutamine (Glut) in the NM (upper) channel).

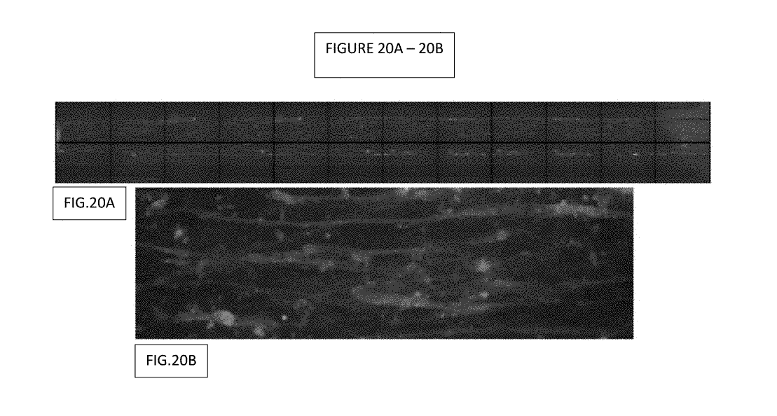

FIG. 20A-20B: Shows exemplary High Content Imaging as immunohistochemistry of iPSC derived Myo-fibers, on fixed cells (Experiment 5).

FIG. 20A: shows a fluorescent micrograph of the entire width and length of immunostained cells in a microfluidic NMJ chip. α-bungarotoxin BTX (green), Neuron-specific Class III β-tubulin (TuJ1) (red) and myosin heavy chain (MHC) (blue).

FIG. 20B: shows a higher power fluorescent micrograph of the channel in the chip shown in FIG. 20A.

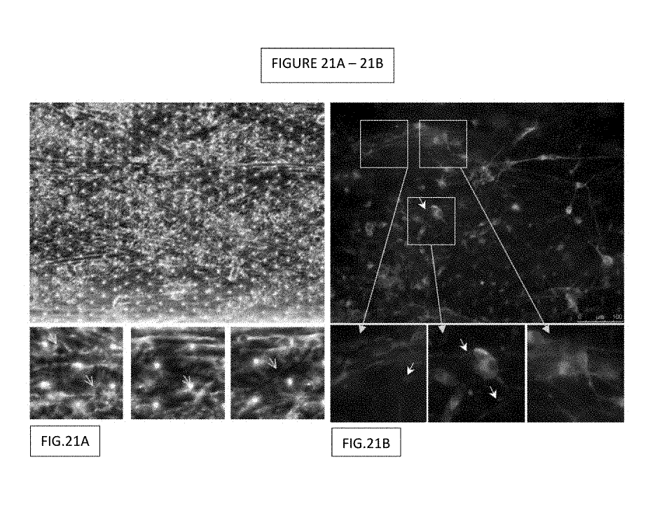

FIG. 21A-21B: shows micrographs of cells grown as shown in Experiment 5 for pharmacology and in-chip imaging for NMJ-On-Chip.

FIG. 21A: shows phase contrast micrographs of myotubes and neurons in chips, higher magnified areas are shown below the larger micrograph white arrows point to potential NMJs where myotubes are adjacent to neurons.

FIG. 21B: shows fluorescent micrographs of superimposed (co-localized images) of neurons stained with a neuronal microtubule marker, Tau, (green) a microtubule stabilization protein, for identifying neurons and motor end plates with BTX (red) (labeling AChRs) for identifying NMJs, where neuronal branches co-localize with end plates. Smaller micrographs show higher magnified areas outlined by corresponding white boxes. White arrows point to motor end plates of myotubes, some of which are in close proximity to neuronal axons.

FIG. 22AA-22CC: shows an exemplary method of growing motor neurons in a microfluidic chip where the MN cells of neural networks have spontaneous calcium bursts. Experiment 5.

FIG. 22AA: shows a microfluidic chip seeded with MNs at day 12 of culture.

FIG. 22BB: shows an exemplary timeline where MN precursor cells from Day 12 cultures are seeded at Day 0 in the microfluidic chip, MN network formation is observed a Day 10 on the chip (Day 18 overall from the start of the original MN culture).

FIG. 22CC: shows exemplary images produced by high content life imaging of cells in chips showing Ca++ imaging of diMN cells on Day 12 after seeding onto the microfluidic chip; at high magnification (20×). diMNs show repetitive calcium bursts as visualized via Flou4 labeling in color within the cellular areas, e.g. cell bodies, axons and terminal bulbs, in neuronal networks, where the concentrations of Ca++ are shown by yellow-lower levels, red-higher than yellow areas and highest levels in white areas within the red areas, as shown in the neuron cell bodies.

FIG. 22A: shows exemplary Ca++ imaging of FIG. 22CC in black and white, where the highest amounts of Ca++ are white areas in black and white micrographs, white arrowheads point to cellular areas with concentrated Ca++.

FIG. 22B: shows a higher magnification of a cell in the center of the micrograph in FIG. 22CC/FIG. 22A with two white arrowhead markers used to identify the same area through the different planes of focus.

FIGS. 22D-22J: shows exemplary Ca++ imaging in color from confocal high content micrograph z-stack layers through the cell (shown in FIG. 22B) where higher concentrations of Ca++ are shown by yellow/red/white areas in the neuronal cytoplasm, which discharge and recharge then discharge over time. White arrowheads mark the same location of the cell shown in FIG. 22B-FIG. 22J.

FIG. 22K: shows a graph of average intensity of Ca++ vs. elapsed time (seconds).

### DETAILED DESCRIPTION OF MICROFLUIDIC CHIPS

It is not intended that the present invention be limited by the nature of the “microfluidic device” or “chip.” However, preferred microfluidic devices and chips are described in U.S. Pat. No. 8,647,861, hereby incorporated by reference, and they are microfluidic “organ-on-chip” devices comprising living cells in microchannels, e.g. cells on membranes in microchannels exposed to culture fluid at a flow rate. It is important to note that the features enabling the actuation of strain or mechanical forces on the cells within the “organ-on-chip” device are optional with regards to the “NMJ-on-chip” and may be omitted.

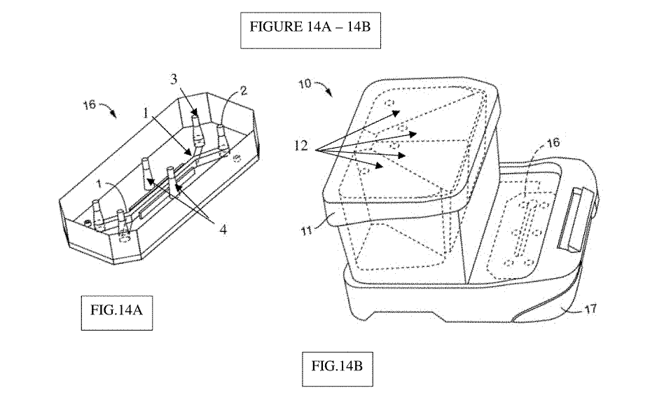

FIG. 14A-14B: Shows schematic illustrations of embodiments of a microfluidic device.

FIG. 14A: is a schematic illustration showing one embodiment of the microfluidic device or chip (16), comprising two microchannels (1), each with an inlet and outlet port for the upper channel (2) and lower channel (3), as well as (optional) vacuum ports (4).

Microfluidic devices are conveniently made of polydimethylsiloxane (PDMS), polyurethane, polycarbonate, polystyrene, polymethyl methacrylate, polyimide, styrene-ethylene-butylene-styrene (SEBS), polypropylene, or any combinations thereof. The present invention contemplates treatment of such substances to promote cell adhesion, selection or differentiation or fluid wetting such as treatments selected from the group consisting of plasma treatment, ion treatment, gas-phase deposition, liquid-phase deposition, adsorption, absorption or chemical reaction with one or more agents.

FIG. 14B: is a topside schematic of an embodiment of the perfusion disposable or “pod” (10) featuring the transparent (or translucent) cover (11) over the reservoirs (12), with the chip (16) inserted in the carrier (17). The chip can be seeded with cells and then placed in a carrier for insertion into the perfusion disposable or pod, whereupon culture media in the reservoirs flows into the microchannels and perfuses the cells (e.g. both MNs and hSMCs).

In one embodiment, the microchannel comprises a surface comprising a silicone polymer. In one embodiment, the silicone polymer is polydimethylsiloxane or “PDMS.” In one embodiment, the ECM protein is covalently coupled to a PDMS surface using a crosslinker.

In one embodiment, one or more proteins (e.g. ECM proteins) or peptides (e.g. RGD) are covalently coupled to the surface of a microchannel of a microfluidic device.

It is not intended that the present invention be limited to any particular protein or peptide; a variety are contemplated, including mixtures. For example, in one embodiment, the covalently attached protein is laminin or collagen. In another embodiment, a mixture of proteins are covalently attached, e.g. a mixture of collagen type I, fibronectin and collagen type IV. In yet another embodiment, the RGD peptide is attached (or a peptide comprising the RGD motif is attached).

In one embodiment, the present invention contemplates a method of culturing skeletal muscle cells, comprising: a) providing a microfluidic device comprising a microchannel comprising a surface, said microchannel in fluidic communication with a fluid source comprising fluid; b) covalently attaching one or more proteins or peptides to said microchannel surface so as to create a treated surface; c) seeding viable skeletal muscle cells on said treated surface so as to create attached cells; c) flowing fluid from said fluid source through said microchannel so as to create flowing conditions; and d) culturing said attached cells under said flow conditions such that said cells remain attached and viable.

It is not intended that the present invention be limited by the manner in which the proteins or peptides are covalently attached. In one embodiment, a crosslinker is used. In another embodiment, a bifunctional crosslinker is used.

A variety of such crosslinkers are available commercially, including (but not limited to) the following compounds:

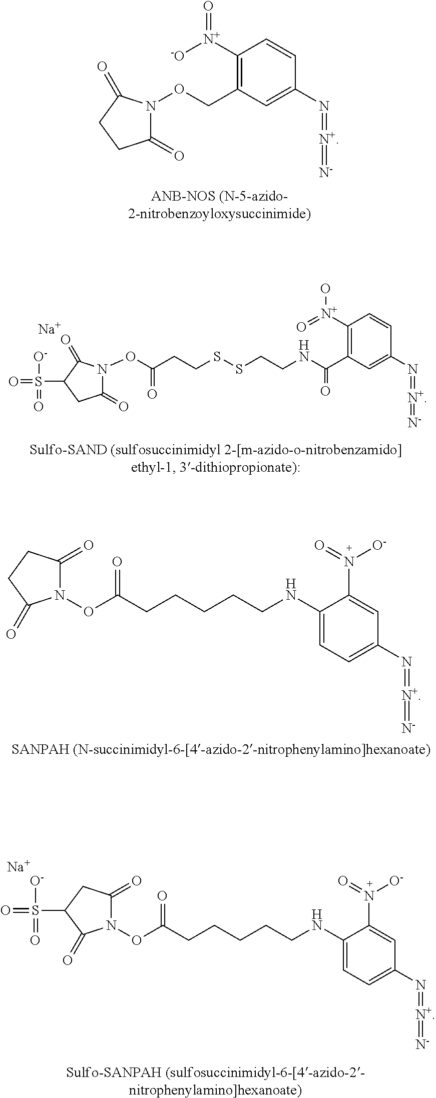

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

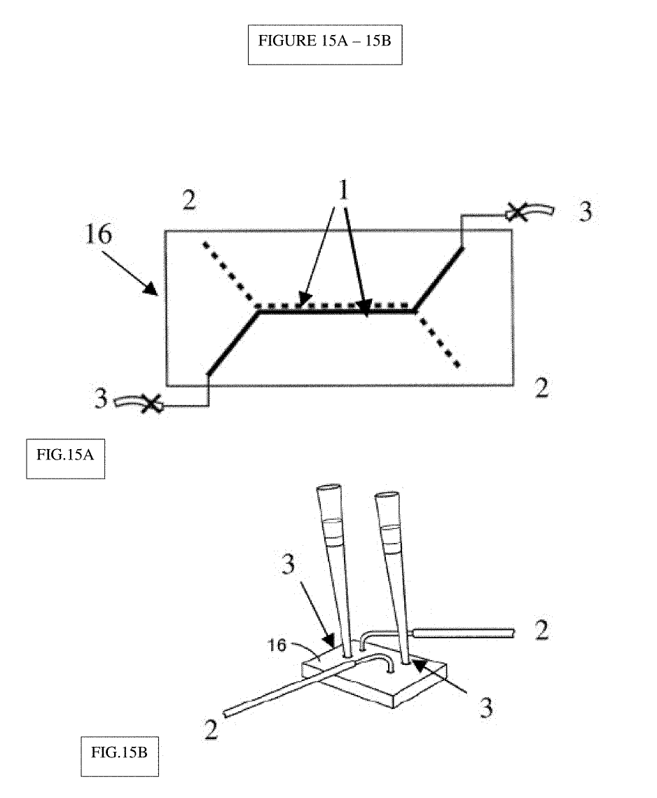

While a variety of protocols were explored, one embodiment of a method for preparing and seeding a microfluidic chip comprises: first, the chip (or regions thereof) are treated to promote wetting or protein adhesion (e.g. by plasma treatment). Second, one or more channels are then plugged (see the top schematic of FIG. 15A, where an “X” indicates a channel is blocked in a microfluidic device or chip with top and bottom channels). FIG. 15B shows how the ports of a microfluidic device can be utilized to introduce fluid (e.g. with ECMs) or cells using pipette tips. Using the protocol, the ECM mixture for the bottom channel is introduced before coating the top of the membrane, with the excess removed, and the remainder dried. Thereafter, the ECM for the top channel is introduced. The hSMCs can be seeded on the bottom channel. The top channel can be washed. Finally, the neural cells can be introduced and incubated for attachment.

The surfaces of the microchannels and/or the membrane can be coated with cell adhesive, selective or promotive molecules to support the attachment of cells and promote their organization into tissues. Where a membrane is used, tissues can form on either the upper surface of the membrane, the lower surface of the membrane, any of the surfaces of the channels or cavities present on either side of the membrane or any combination thereof.

FIG. 15A-15B: Shows schematic illustrations showing one embodiment of microfluidic devices, including for providing an “air dam” for isolating one channel.

FIG. 15A: is a schematic illustration showing one embodiment of a microfluidic device or chip (16) (viewed from above), the device comprises top (apical; dotted line) and bottom (basal; solid line) channels. As an example, motor neurons are seeded into the upper (apical) channel and human skeletal muscle cells are seeded into the lower (basal) channel. In one embodiment, an “air dam” is created for part of a protocol, described below, where the two Xs are indicating that channels are blocked during at least part of the protocol.

FIG. 15B: is a schematic illustration showing one embodiment of how ports, upper (2) and lower (3) of a microfluidic device or chip (16) can be utilized to deposit fluids carrying surface coatings (e.g. dissolved proteins) and/or seed the cells using pipette tips. This image, in part, shows one embodiment of a modification to the typical chip ECM coating protocol based on the need in some embodiments to coat the top and/or bottom channels with different ECM solutions in wet and/or dry conditions.

In one embodiment, the upper channel port (2) is blocked, while ECM or cells are added to the lower channel port (3).

The procedure developed involved an “air dam” by which perfusion of ECM1, for example, loaded into the top channel (apical; dotted line) was prevented from perfusing through the membrane to the bottom channel (basal; solid line) by clamping flexible tubing and trapping air in the bottom channel, FIG. 16A. The ports of a second microfluidic channel can be air-filled and plugged up using clips, for example. For covering the surface of the lower channel, the ports (2) for the top channel are plugged for preventing perfusing of ECM, such as laminin, through the membrane into the upper channel.

In one embodiment, different cells are living on the upper and lower surfaces, thereby creating one or more tissue-tissue interfaces separated by the membrane. The membrane may be porous, flexible, elastic, or a combination thereof with pores large enough to only permit exchange of gases and/or small chemicals, or large enough to permit migration and transchannel passage of large proteins, as well as whole living cells and/or portions thereof (e.g. forming neuronal terminal synapses with muscle cells). Depending on the size-scale of the pores and manufacturing preferences, the pores may be defined, for example, using lithography, molding, laser-drilling or track-etching, intrinsic to a selected material (for example, polyacrylamide gel, collagen gel, paper, cellulose) or engineered into the material (e.g. by generating an open-cell polymer or matrix).

Flow is important and stands in contrast to static 2D culture. Using a flow in the microchannel(s) allows for the perfusion of cell culture medium throughout the cell culture during in vitro studies and as such offer a more in vivo-like physical environment. In simple terms, an inlet port (2 and 3) allows injection of cell culture medium, test agents, etc. into a cell-laden microfluidic channel (1) or chamber (1), thus delivering nutrients and oxygen to cells. An outlet port (2 and 3) then permits the exit of remaining liquid as well as harmful metabolic by-products. While continuous flow is preferable due to its application of controlled shear forces, either of the device's fluidic paths could also be cultured under “stop flow” conditions, where the flow is engaged intermittently, interspersed by static culture.

It is not intended that the present invention be limited to particular “flow rates” or means for generating flow rates. In one embodiment, a flow rate of between 5 and 200 uL/hr, and more preferably between 20-100 uL/hr, and still more preferably between 10 and 60 uL/hr, and still more preferably between 20-50 uL/hr, is contemplated. In one embodiment, pressure is applied through the lid and the lid seals against the reservoir(s). For example, when one applies 1 kPa, this nominal pressure results, in one embodiment, in a flow rate of approximately 30-40 uL/hr. When one applies a pressure of between 0.5 kPa, this nominal pressure results, in one embodiment, in a flow rate of between 15 uL/hr and 30 uL/hr.

In one embodiment, a tall 2 chamber (upper and lower) PDMS microfluidic Chip has a membrane separating the two chambers having a pore diameter of 7 μm, spacing: 40 μm Hex packed, thickness: 50 μm, extracellular matrix (ECM) provided is laminin (250 μg/ml).

### EXAMPLES

### Example 1

In this example, several exemplary embodiments are provided for the generation of motor neurons is provided using iPSCs as the starting material, see, Table 1 and Table 2. In one embodiment, a MN-on-chip is provided with MNs seeded into the upper channel of a microfluidic chip. In another embodiment, MNs are seeded into the upper channel of a NMJ-On-Chip.

Cells are prepared either directly from cultured iPSCs or from frozen lots of pre-differentiated cells. Cells are thawed (or dissociated fresh) and seeded into the chip at day 12 (in the case of iMN differentiation) and at various points in neural differentiation.

More specifically, for example, MN cells are seeded at day 12 of differentiation either from freshly differentiated cultures or directly from a thawed vial into a microfluidic chip described herein.

CALCIUM FLUX: FIG. 22AA-22CC show the results of calcium flux imaging in the upper neural channel. Using a florescent calcium influx-activated dye (Fluo-4), neurons seeded in chip were imaged using a high-resolution high frame-rate camera. Florescence intensity changes of up to hundreds of neurons were analyzed simultaneously by recording average pixel intensity over time (dF/F). These values were plotted with respect to time and are analyzed for waveform properties, which correlate spontaneous neural activity and neural network formation. This is accomplished through multi-step video post-processing and signal analysis (including video compression, signal cleanup, automatic or manual ROI detection, etc. which can be implemented from open-source MATLAB software packages). The photograph (FIG. 22CC) is a single fluorescent image from a movie of such images. The colored areas (yellow, red and white within red areas) indicate areas of Ca++ hot spots, i.e. higher concentrations of Ca++. The addition of tetrodotoxin (TTX), which is a potent blocker of voltage-gated calcium channels, ablates this activity. This type of experiment is contemplated to show neuronal activity modulated by pharmacological stimulation.

In a controlled study, live cell imaging was performed on diMNs that had been cultured in the chip (MN-on-Chip) (FIGS. 22BB-22J). High content imaging of neuron calcium flux was recorded and plotted with respect to time (FIG. 22K). Calcium flux events or peaks correspond to neural activity and were counted by both automated software and blinded human technician. Each event was assigned a time-stamped value and depicted for each tracked neuron with respect to time. This Calcium (Ca++) flux live cell assay showed Ca flux in relation to spontaneous neuronal activity, i.e. firing. For examples, see FIG. 22AA-22CC.

### Example 2

In this example, several exemplary embodiments are provided for the generation of hSkMCs on microfluidic chips for skeletal muscle cells-on-chips (and then for NMJ-On-Chips), using myoblasts and/or iPSCs as the starting material.

The following describes exemplary methods, e.g. for differentiating iPSCs, providing a Muscle Cell Culture-on-Chip.

Skeletal Muscle Differentiation from Human iPSCS.

The starting density of cells affects the success of skeletal muscle cell differentiation. The starting iPSc density described herein is exemplary for the cell lines described herein. However each iPSC line is different so the optimal density should be determined according to each individual cell line's growth (e.g. doubling) rate. For cell lines shown herein, an exemplary recommended cell density and volume of media: 12 or 24 wells 15,000-18000 cells/cm² and for 96 wells 5000 cells/cm². One embodiment for a method providing human induced pluripotent stem cells (iPSCs) for use in providing induced hSkMCs is described as follows.

Coat plates with ECM, e.g. Matrigel. Add appropriate volume, see e.g. below, in a sterile tissue culture hood. For a 6 well plate—1 mL/well; 24 well plate—250 μL/well; and 96 well plate—50 uL/well. Leave Matrigel in wells for at least 1 hr at room temperature for coating surfaces. Coating may also be done for more than an hour.

For deriving human iPSC (hiPSC) skeletal cell cultures from hiPSCs: Grow and expand iPSC cultures on Matrigel coated surfaces with mTeSR Media supplemented with Rock Inhibitor (Y-27632) (such as from Sigma-Aldrich, St. Louis, Mo. 63103-USA), at exemplary concentrations from 2.0 uM, 2.5 uM, 5 uM, 10 uM, up to 20 uM, for one day. Nonlimiting examples of mTeSR Media include, cGMP mTeSR™1, mTeSR™1, TeSR™2, TeSR™-E7™, TeSR™-E5, TeSR™-E6, ReproTeSR™, mTeSR™3D, etc., defined, serum-free media for culture of human ES, iPS, pluripotent stem cells, and the like). Clean iPSCs cells daily by removing differentiated cells to maintain a spontaneous differentiation free culture for optimal skeletal muscle differentiation. In one embodiment, 3 wells of a 96 well plate containing iPSCs, maintained at 70-80% confluence is suggested for use to start differentiation.

More specifically, Stage 1 skeletal muscle induction: Step 1. Dissociate iPSCs with Accutase (e.g. of a cell detachment solution) for 5 min.; Step 2. Resuspend cells in phosphate buffered saline (PBS) in a 15 mL conical tube; Step 3. Centrifuge the cells for 5 min (minutes) at 1000 RPM (revolutions per minute) for spinning cells gently to the bottom of the tube; Step 4. Aspirate media without disturbing the cell pellet in the bottom of the tube, then resuspend cells in skeletal muscle induction media 1, DMEM/F12, (see, Table 3); Step 5. Count the number of live cells (in part by exclusion staining the dead cells), e.g. using an automated cell counter: Take out 10 ul of cell suspension from the tube, mix with 10 ul of dye (1:1), e.g. in Trypan blue dye for staining dead cells, mix well, load mixture in cell counter chamber to count; Determine live cell numbers per ml, then Step 6. Plate single cells with appropriate number of cells, as suggested herein, on a Matrigel coated plate in mTeSR Media supplemented with Rock Inhibitor (Y-27632), see exemplary materials and concentrations above, for one day; Step 7. On the next day, switch the Stage 1 media to DMEM/F12 (1:1) supplemented with exemplary concentrations of 3 uM CHIR99021, 0.5 uM LDN193189; Step 8. Change media everyday until day three; then Step 9. On Day three, supplement the existing media with an exemplary concentration of 20 ng/mL bFGF and continue feeding for additional seven days. Media should be change on a daily basis.

Stage 2—Commitment to Myoblasts. 1. After 10 days of incubation (e g. 7 days incubation in complete skeletal muscle induction media 1), the media is changed to a DMEM/F12 (1:1) supplemented with exemplary concentrations of 10 ng/ml HGF, 2 ng/ml IGF and 0.5 uM LDN193189 (Skeletal Muscle Induction Media 2) for two days of incubation, see Table 4; If cells are too confluent by day 12-14, cells should be dissociated and replaced on ECM, e.g. Matrigel coated surfaces at recommended cell densities, mentioned above, for optimal results; and 2. On day 12, cells were cultured with DMEM/F12 (1:1), with exemplary concentrations of 15% KSOR supplemented with an exemplary concentrations of 2 ng/ml IGF (incomplete Skeletal Muscle Induction Media 3), see Table 5; for up to four days.

Stage 3 Maturation: For differentiation of myoblasts into myotubes and for maintenance of skeletal muscles: 1. On Day 12, 13 or 14, media was changed to DMEM/F12 (1:1), with exemplary concentrations of 15% KSOR supplemented with 10 ng/mL HGF and 10 ng/mL IGF-1 (complete Skeletal Muscle Induction Media 3), see Table 5; 2. Change Media every other Day until used, up to day 40; and 3. Optional: Fix cell samples, up to day 40 (or day used), e.g. of fixative, 4% PFA (Paraformaldehyde) to stain for skeletal muscle markers, e.g. as described herein. Other fixatives may be used for immunostaining.

The exemplary protocol described here for differentiating hSkMCs was used on ECM coated substrates, such as plates and microfluidic channels. For examples of ECM, plates and channels were coated with Matrigel, while microfluidic channels were coated with Laminin (non-cross-linked) and cross-Linked Laminin, as described herein. Seeding densities for the chips were used as described for the experiments, where either ihSkMCs were differentiated as described here, as one example, starting myotube differentiation on D1 in Stage 1 Skeletal Muscle Induction Media (incomplete).

### Example 3

In this example, several exemplary embodiments are provided for the generation of hSkMCs on microfluidic chips coated with ECM for testing Extracellular Matrix effects on myotube structure and stability.

A. Extracellular Matrix (ECM).

In some embodiments, an extracellular matrix (ECM) layer is provided to coat (cover) the entire surface (bottom, sides and top) of the lower channel for growing human skeletal striated muscle cells. In one embodiment, Laminin was used as an exemplary ECM component for coating the surface. In another embodiment, a cross-linker chemical was used for cross-linking Laminin molecules. As an exemplary cross-linker chemical, Sulfo-SANPAH was used.

FIG. 4A-4C: Shows one embodiment of a human skeletal muscle cell culture hSkMC-In-Chip: Extracellular Matrix (ECM) use for hSkMCs-In-Chip. In one embodiment, the chip is a Quad chip.

FIG. 4A: shows a picture of a single channel (Quad) Chip with pipette tips used to block channels for coating the inside surfaces with an ECM layer then seeded with human skeletal muscle cells (hSKMCs).

FIG. 4B: shows a schematic illustration of a cross-sectional view of the quad channel with ECM as Laminine (purple and blue stars) with hSkMCs as yellow-spotted blocks.

FIG. 4C: shows a schematic illustration of a cross-sectional view of the quad channel with ECM as Laminine (purple and blue stars) with hSkMCs as yellow-spotted blocks and a representative cross linking of ECM as yellow stars, e.g. with Sulfo-SANPAH.

B. Extracellular Matrix (ECM) Cross-Linking Effects on Myotube Structure and Stability in Chips.

This example shows one embodiment of a set up and time course for culturing Human Muscle Cells in-Chip: providing non-contracting myotubes on ECM coated chips. As one embodiment, a single channel chip (e.g. Quad chip: as a 4 single channel chip) was used initially for determining stages of muscle cell maturation on a chip, effects of ECM, and numbers of seeded cells that provide viable cultures in relation to chips coated with ECM. In this embodiment, muscle cells grown without nerve cells present did not show spontaneous contractions of myotubes.

Experiment 2 showed that Sulfo-SANPAH cross linked ECM provides more stability to hSkMCs. As one example, Sulfo-SANPAH cross-linked ECM enables formation of almost 2-fold more MHC positive multinucleated fibers. Further, more nuclei per myo-tubes with cross-linked ECM. In fact, a 3-fold higher number of nuclei in MHC myo-fibers seeded on Sulfo-SANPAH cross-linked ECM-Laminin was observed over a Laminin coating without the use of a cross-linker.

FIG. 5A-5D: shows one embodiment of a human muscle cell culture in-chip: Set Up and Time Course for producing multinucleated myofibers that are not contracting.

FIG. 5A: Single channels of Quad Chips were seeded with human skeletal muscle cells (hSKMCs). Group 1 and Group 2: 5×10⁶/ml cells; Group 3 and Group 4: 1.6×10⁶/ml cells. Groups 1 and 3 do not have cross (X)-linked ECM while Groups 2 and 4 have exemplary Sulpho-SANPAH X-linked ECM.

FIG. 5B: shows a schematic experimental timeline: Seeding cells on Day (D) 0. D1: Inducing differentiation. D5 observing fusion of myoblast cells. D10: Screening for myo-fiber contraction in cultures that were not stained for analysis; observing polynucleated fibers but no myofiber contractions. D14 Fixing cells and fusion-index-analysis.

FIG. 5C: Day 14: Fixation and fusion-index-analysis based upon staining for myosin heavy chain (MHC) (red) and nuclei (DNA) (shown in blue).

FIG. 5D: Shows a schematic illustration of multinucleated myofibers MHC (red) and nuclei (DNA) (blue).

FIG. 6A-6G: shows Human Skeletal Myoblast-Derived Poly-Nucleated Fibers growing in microfluidic chips where Sulfo-SANPAH cross-linked ECM enables formation of almost 2-fold more MHC positive multinucleated fibers.

FIG. 6A-6D: show fluorescent micrographs of immunostained myosin heavy chain (MHC) (red) myo-fibers and DAPI stained nuclei (DNA) (shown in blue) comparing cultures started at the 2 different densities (FIGS. 6A-B: 5×10⁶/ml cells and FIGS. 6C-6D: 1.6×10⁶/ml cells) with and without cross-lined (X-link) ECM-Laminin (Lam).

FIGS. 6E-6F: show phase contrast micrographs of Day 14 cells grown on Laminin (Lam) and cross-linked (X-Link) ECM-Laminin (Lam), respectively. More MHC positive multinucleated fibers are observed with X-Linked Laminin after 14 days. White arrows point to 2 exemplary multinucleated myotubes

FIG. 6G: shows a graph comparing number MHC+ myo-fibers to the treatments shown in FIGS. 6A-6D where at both cell densities the number of myofibers growing on x-Linked ECM is almost 2-fold more than fibers grown on regular, non-cross-linked, ECM.

FIG. 7A-7F: shows Human Skeletal Myoblast-Derived Poly-Nucleated Fibers growing in microfluidic chips comparing non-cross-linked to cross-linked ECM (Laminin) where more nuclei per myo-tubes are observed growing on cross-linked ECM.

FIG. 7A-7D: show fluorescent micrographs of immunostained myosin heavy chain (MHC) (red) myo-fibers and DAPI stained nuclei (DNA) (shown in blue) comparing cultures started at the 2 different densities with inserts showing higher magnifications of presumptive myo-fibers for each treatment.

FIGS. 7A-7B: 5×10⁶/ml cells and FIGS. 6C-D: 1.6×10⁶/ml cells) with Laminin (Lam) and with cross-linked (X-linked) Laminin-ECM.

FIGS. 7E-7F: Show a 3-fold higher number of nuclei in MHC myo-fibers seeded on exemplary Sulfo-SANPAH cross-linked ECM by graphical comparisons.

FIG. 7E: shows a graph comparing DAPI+ nuclei per MHC+ fiber for determining myo-fiber at the 4 treatments shown.

FIG. 7F: shows a graph comparing percentage of total DAPI+ per channel, i.e. percentage of DAPI in myo-fibers at the 4 treatments shown in FIG. 7A-D.

### Example 4

In this example, exemplary embodiments are provided for a Human iPS-Derived MN and Muscle Cell Co-Culture in-Chip showing a loss of myotubes starting around 24 hours after start of spontaneous contractions.

Experiment 1: Human iPS-Derived MN and Muscle Cell Co-Culture in-Chip.

Day 0: seeding hSkMCs; Day 1: (18 h later) seeded diMNs (D12); Day 5: observation of formation of myotubes; Day 10: observation of myofiber contraction; Day 11: observation of progressive loss of myofibers; Day 14: fixation and analysis. There was a continuous loss of myo-tubes after day 11-24 hours, after last observation of spontaneous myo-tube contractions. Further, the use of flow during culture increases loss of myo-tubes. See, FIG. 9A-9C for a schematic illustration and numbers of cells in the different replicates for comparing effects of initial seeding densities.

Experiment 1 showed that hSkMC seeding density at 3×10⁶ cells/ml, but loss of cells 24 h after contracting activity.

FIG. 9A-9C: Shows one embodiment of a Human iPS-Derived MN and Muscle Cell Co-Culture in-a microfluidic Chip.

FIG. 9A is a picture of an exemplary microfluidic chip where day 12 MNs are seeded into the top (upper-blue) channel and hSkMCs are in the bottom (lower-red) channel;

FIG. 9B shows a schematic illustration of an exemplary cross section of NMJ microfluidic chip with day 12 MNs in the top channel and hSkMCs in the bottom channel with 3 sets of Experimental Chips for comparing cell densities at the time of seeding: Chip 1: top: 3×10⁶/ml diMN cells and bottom: 5×10⁶/ml hSkMC cells; Chip 2: top: 3×10⁶/ml diMN cells and bottom: 10×10⁶/ml hSkMC cells; and Chip 3: top: 3×10⁶/ml diMN cells and bottom: 20×10⁶/ml hSkMC cells.

FIG. 9C: shows a schematic illustration of a timeline showing co-culture of hSkMCs seeded Day (D) 0 with differentiation (diff) initiated on D1, Day 12 MNs seeded D1, Myofiber formation on D5, myofiber contractions observed D10, a loss of myofibers observed on D11, with fixation and analysis by ICC on D14.

### Example 5

This example describes one embodiment of method steps for providing a functional NMJ-on-chip with reduced spontaneous myotube contractions. The following experiments were designed for identifying media components that would lower spontaneous contraction rates.

Media was tested that included at least one agent for reducing spontaneous myotube contraction rates. In part, rates were artificially reduced in order to allow testing of agents for altering muscle contractions, e.g. increasing muscle contraction rates.

By day 10 of cultures, observations of myotubes showed high rates of spontaneous contractions. Therefore, experiments were designed for identifying media that would reduce spontaneous contractions in cultures.

FIG. 10A-10B: shows one embodiment of an experimental system (Experiment 1) as a schematic illustration for testing medium to reduce spontaneous contractions of cells in the microfluidic tall channel chip. Experimental Groups 1-3 directly compare medium harvested from diMNs/hSkMC cultures with coM media in chips containing induced motor neurons (diMNs: Motor-neuron-on Chip) and human Skeletal Muscle Cells (hSkMCs-on-Chip), each cell type growing alone on chips then combined in the same chip in the same media (upper and lower channel) for providing a neuronal-muscular-junction (NMJ-on-Chip).

FIG. 10A: Group 1: shows a schematic illustration of the tall channel chip, with vacuum chambers (4), diMNs in the top channel but no cells in the bottom channel. Group 2: shows a schematic illustration of the tall channel chip with no cells in the top channel but with hSkMCs in the bottom channel. Group 3: shows a schematic illustration of the tall channel chip with diMNs in the top channel and hSkMCs in the bottom channel for providing a NMJ-on-Chip.

FIG. 10B: shows a schematic illustration of cells numbers and media used for growing cells: Group 1: Top: 3×10⁶ diMNs Bottom: none. Group 2: Top: none. Bottom: 10×10⁶ hSkMCs. Group 3: Top: 3×10⁶ diMNs. Bottom: 20×10⁶ hSkMCs.

FIG. 11A-11C: Shows human skeletal muscle cells (hSkMCs) forming myofibers within 8 days post seeding (co-cultures) having spontaneous myo-tube contractions at Day (D) 10 culture that are reduced by using coal culture medium in a microfluidic chip.

FIG. 11A: shows micrographs of hSkMCs growing in chips. White arrows in the magnified region point to multinucleated muscle cell fibers, of which there appears to be more nuclei per fiber in the coM medium;

FIG. 11B: shows micrographs of diMNs growing in chips; and

FIG. 11C: shows micrographs of shSkMCs/diMNs grown in MN/hSkMCs media (upper row of micrographs) and coM medium (lower row of micrographs) growing in chips. Spontaneous myotube contraction was observed only in diMNs/hSkMC co-cultures. White arrows in the magnified region point to contacts of MN with a muscle cell fiber.

Inserts show higher magnified areas of cells outlined in the white box for each micrograph.

FIG. 12A-12D: Shows human skeletal muscle cells (hSkMCs) as myofibers with spontaneous myotube contraction at Day (D) 10 (Experiment 3).

FIG. 12A: shows a micrograph of hSkMCs as myotubes growing on top of a membrane of the microfluidic chip in coM media.

FIG. 12B: shows a graph comparing contractions per minute for a myofiber contraction frequency with an average of fibers in two experiments (Experiment 1 and 3) that were combined for a total estimation of myofiber contraction frequency.

FIG. 12C: shows a graph comparing contractions per minute for myofibers having an increased myofiber contraction frequency between Laminin vs. cross linked Laminin ECM, at about the same frequency as shown in FIG. 12B.

FIG. 12D: shows a graph comparing contractions per minute for myofibers grown in regular media compared to a culture grown in coM media. When cultured in coM, contraction frequency is around 25% less compared to regular medium conditions.

FIG. 13A-13B: shows schematic illustrations of experimental timelines for comparing co-cultures of hSkMCs with MNs, with and without coM media.

FIG. 13A: shows a schematic illustration of a timeline and cell densities for Group 1 and Group 2 in coM: hSkMCs seeded at 5×10⁶/ml cells and MNs seeded at 3×10⁶/ml cells. hSkMCs seeded Day (D) 0 with differentiation (diff) initiated on D1. Day 12 MNs seeded D1 (as one example 18 h later), D5 formation of myotubes & medium switch to coM, no myofiber contractions observed D10, no loss of myofibers observed on D12, fixation and analysis by ICC on D14, duplicate chips on D20 showed no loss of myofibers.

FIG. 13B: shows a schematic illustration of a timeline and cell densities for Group3: hSkMCs seeded with MNs: Day 0: seeding hSkMCs; Day 1: (18 h later) seeded diMNs (d12); Day 5: formation of myotubes, no medium switch; Day 10: observation of myofiber contraction; Day 11: observing progressive loss of myofibers; Day 14: fixation and analysis by ICC; in chip cultures left to D20, there is almost a complete loss of myofibers.

Thus, exemplary steps for providing a functional NMJ-on-Chip by combining motor-neurons on a chip (upper blue channel) with skeletal muscle cells on a chip (lower-red) channel include: Seeding the bottom (lower-blue) channel as a skeletal muscle-on-chip capable of producing contractile muscle tissue expressing markers myosin heavy chain (MHC) (green), pre-BTX (α-bungarotoxin) (red) identified by immunohistochemistry and stained for DNA (blue) shown by fluorescent microscopy. Seeding the upper channel of the microfluidic chip with patient iPSC-derived MNs that under chip culture conditions will express neuronal expressing markers Neuron-specific Class III β-tubulin (TuJ1) (red), selectivity/elective factor 1 complex (for RNA polymerase) (SL1) (blue), homeobox B9 (HOXB9) (red), identified by immunohistochemistry (IHC) as shown by fluorescent microscopy. In some embodiments, spontaneous contractions may be stopped by adding calcium channel blockers or sodium channel blockers to the culture media.

### Example 5

This example shows embodiments of exemplary co-localization of MNs and muscle cells showing potential formation of NMJs in microfluidic NMJ-on-chip.

FIG. 14A-14B: shows florescent micrographs of stained cells in a microfluidic chip. Co-Localization Study of iPS-Derived MNs and Muscle Cells. Both diMNs and hSkMCs are in close proximity to each other as determined from initial ICC analysis and 3D reconstruction of confocal microscope images (i.e. combined z-stacks). A partial loss of myotubes were observed due to lack of ECM stability

FIG. 14A and FIG. 14B: α-bungarotoxin (BTX) for identifying the motor end plate (green), skeletal muscle marker, desmin, (red) and DNA (DAPI) (shown in blue). The red muscle fiber is multinucleated with numerous green motor end plates.

FIG. 14B: a higher magnification of FIG. 14A, 3 white arrows point to co-localization of α-bungarotoxin (BTX) for identifying the motor end plate (green) and skeletal muscle marker, desmin, (red) as olive, white dark orange areas depending upon concentration of stain.

FIG. 14C and FIG. 14D: motor end plate (green) BTX and neurofilament H nonphosphorylated (SMI 32) (red) and DNA (DAPI) (shown in blue).

FIG. 14D: a higher magnification of FIG. 14C, 3 white arrows point to co-localization of a motor end plate (green) BTX, neurofilament H non-phosphorylated (SMI 32) (red) as olive-white areas depending upon concentration of stain.

### Example 6

This example describes using Microfluidic NMJ-On-Chip Under Flow For Longer Studies.

### Experiment 4: Extended Cultures up to Day 37

Experiment 4 showed that hSkMC integrity in chip is expandable over time (in monoculture).

FIG. 17A-17G: shows schematic illustrations of tall channel microfluidic NMJ-on-chip with one embodiment of an experimental timeline (Experiment 4) set up and time course for comparing co-cultures of hSkMCs with MNs under flow.

FIG. 17A: shows a schematic illustration of a tall channel microfluidic chip, from left to right, view of vertical 2-channel chip (i.e. the top channel is above the bottom channel as shown in Stage 1, with hSkMCs covering the entire surface of the bottom channel, and Stage 2 with diMNs seeded into the top channel.

FIG. 17B: shows a schematic illustration of one embodiment of a timeline where hSkMCs are seeded Day (D) 0 with differentiation (diff) initiated on D1, D 5: formation of myotubes & medium switch to coM media, then Day 7-10: no myofiber contraction, on Day 20 start muscle cells under flow at 10 ul/hour, continued to D29 when flow is stopped. Day 30: seed diMNs (d12) (not in coM media for observing baseline contractions). Day 37: myotubes are spontaneously contracting: fixation and analysis (including ICC).

FIG. 18A-18D: shows an exemplary co-localization study of iPS-Derived MNs and Muscle Cells showing formation of NMJs between diMNs and hSkMCs (Experiment 4). Cells were stained with α-bungarotoxin (BTX) for identifying suggestive NMJ areas where motor end plate (green), neurons are stained with Tubulin beta-3 chain (Tubb3) (red) and muscle myosin heavy chain (MHC) (blue) were fluorescently imaged on individual channels then merged. The blue channel of MHC staining is not shown in FIG. 12A-12D.

FIG. 18A: shows a low power fluorescent micrograph where Tubb3 (red) neuronal staining shows neurite extension along myotubes with oval areas (green) suggestive of lower motor nerve termini whose distribution over a myotube suggests motor end plates.

FIG. 18B-G: shows higher power fluorescent micrographs of the suggestive NMJ areas (white arrows) are identified by superimposed staining i.e. co-localization, where the red stained nerve terminal neuron bulb is co-localized with BTX green staining of motor end plates producing a yellow NMJ.

FIG. 18E-18G: The blue channel of MHC staining is shown showing a MHC containing muscle fiber at the yellow stained NMJ.

### Example 7

In this example a microfluidic NMJ-on-chip described for pharmacology studies and live imaging of cells within channels (Experiment 5).

In this embodiment, an experimental time line (course) is described for seeding hSkMCs up to 9 days prior to seeding MNs in the upper channel. Spontaneous contractions are allowed to begin by removing CoM media at the start of the pharmacology assay.

Experiment 5 showed that pharmacology and imaging was possible for measuring functional NMJ interactions.

FIG. 19A-19B: shows schematic illustrations of one embodiment of experimental timelines for using NMJ-on-chips (Experiment 5) as a set up and time course for using co-cultures of hSkMCs with MNs for live imaging and pharmacology studies.

FIG. 19A: shows a schematic illustration of a tall channel microfluidic chip, seeded with hSkMCs at Day 0 (D0) in the bottom channel, culting up to D9, without observing muscle contractions, then D9 seeding diMNs (d12). In one embodiment only in Group 2. In some embodiments, more than one group of hSkMCs receive MNs. On days 15, 16 and/or 17, live imaging of pharmacology assays are done as shown schematically, for one example, in FIG. 19B.

FIG. 19B: shows a schematic illustration of one embodiment of a timeline where a NMJ-On-Chip with spontaneous contracting muscle fibers is used for a pharmacology study, i.e. testing agents for inducing or reducing muscle contractions on a baseline chip with or without spontaneously contracting myofibers, in one embodiment, treating NMJ chip with 75 uM Glutamine (Glut) in the NM (upper) channel), in one embodiment, treating NMJ chip with 12 uM alpha-turbocurarine in the hSkMC (lower) channel), in one embodiment, washing out alpha-turbocurarine, in one embodiment, treating NMJ chip with 100 uM Glutamine (Glut) in the NM (upper) channel).

FIG. 20A-20B: Shows exemplary High Content Imaging as immunohistochemistry of iPSC derived Myo-fibers, on fixed cells (Experiment 5).

FIG. 20A: shows a fluorescent micrograph of the entire width and length of immunostained cells in a microfluidic NMJ chip. α-bungarotoxin BTX (green), Neuron-specific Class III β-tubulin (TuJ1) (red) and myosin heavy chain (MHC) (blue).

FIG. 20B: shows a higher power fluorescent micrograph of the channel in the chip shown in FIG. 20A.

FIG. 21A-21B: shows micrographs of cells grown as shown in Experiment 5 for pharmacology and in-chip imaging for NMJ-On-Chip.

FIG. 21A: shows phase contrast micrographs of myotubes and neurons in chips, higher magnified areas are shown below the larger micrograph white arrows point to potential NMJs where myotubes are adjacent to neurons.

FIG. 21B: shows fluorescent micrographs of superimposed (co-localized images) of neurons stained with a neuronal microtubule marker, Tau, (green) a microtubule stabilization protein, for identifying neurons and motor end plates with BTX (red) (labeling AChRs) for identifying NMJs, where neuronal branches co-localize with end plates. Smaller micrographs show higher magnified areas outlined by corresponding white boxes. White arrows point to motor end plates of myotubes, some of which are in close proximity to neuronal axons.

FIG. 22AA-22CC: shows an exemplary method of growing motor neurons in a microfluidic chip where the MN cells of neural networks have spontaneous calcium bursts. Experiment 5.

FIG. 22AA: shows a microfluidic chip seeded with MNs at day 12 of culture.

FIG. 22BB: shows an exemplary timeline where MN precursor cells from Day 13 cultures are seeded at Day 0 in the microfluidic chip, MN network formation is observed a Day 10 on the chip (Day 18 overall from the start of the original MN culture).

FIG. 22CC: shows exemplary images produced by high content life imaging of cells in chips showing Ca++ imaging of diMN cells on Day 12 after seeding onto the microfluidic chip; at high magnification (20×). diMNs show repetitive calcium bursts as visualized via Flou4 labeling in color within the cellular areas, e.g. cell bodies, axons and terminal bulbs, in neuronal networks, where the concentrations of Ca++ are shown by yellow-lower levels, red-higher than yellow areas and highest levels in white areas within the red areas, as shown in the neuron cell bodies.

FIG. 22A: shows exemplary Ca++ imaging of FIG. 22CC in black and white, where the highest amounts of Ca++ are white areas in black and white micrographs, White arrowheads point to cellular areas with concentrated Ca++.

FIG. 22B: shows a higher magnification of a cell in the center of the micrograph in FIG. 22CC/FIG. 22A with two white arrowhead markers used to identify the same area through the different planes of focus.

FIGS. 22D-22J: shows exemplary Ca++ imaging in color from confocal high content micrograph z-stack layers through the cell (shown in FIG. 22B) where higher concentrations of Ca++ are shown by yellow/red/white areas in the neuronal cytoplasm, which discharge and recharge then discharge over time. White arrowheads mark the same location of the cell shown in FIG. 22B-FIG. 22J.

FIG. 22K: shows a graph of average intensity of Ca++ vs. elapsed time (seconds).

1. A method of culturing cells, comprising: a) providing a microfluidic device comprising a membrane, said membrane comprising a top surface and a bottom surface; b) seeding induced motor neuron cells on said top surface and skeletal muscle cells on said bottom surface so as to create seeded cells; c) exposing said seeded cells to a flow of culture media for a period of time; and d) culturing said seeded cells under conditions such that a neuromuscular junction forms within said microfluidic device.

2. The method of claim 1, wherein said skeletal muscle cells are induced to differentiate.

3. The method of claim 2, wherein said skeletal muscle cells form contractile tissue.

4. The method of claim 2, wherein said skeletal muscle cells form polynucleated myo-fibers.

5. The method of claim 1, wherein said seeded cells are cultured for more than ten days.

6. The method of claim 1, wherein said induced motor neuron cells are derived from induced pluripotent stem cells from a human.

7. The method of claim 6, wherein said human is diagnosed with a CNS disorder.

8. The method of claim 1, further comprising the step of e) assessing the health and/or integrity of the neuromuscular junction.

9. The method of claim 1, further comprising the step of e) electrically stimulating said motor neurons and/or said skeletal muscle cells.

10. A method of culturing cells, comprising: a) providing a microfluidic device comprising a channel; b) seeding skeletal muscle cells into said channel; c) inducing said skeletal muscle cells to differentiate; and d) detecting myo-fiber formation.

11. The method of claim 10, wherein said detecting of myo-fiber formation comprises detecting myo-fiber contractions.

12. The method of claim 10, wherein said seeded cells are exposed to a flow of culture media for a period of time.

13. A method of culturing cells, comprising: a) providing a microfluidic device comprising a patterned surface and a gel, b) seeding induced motor neuron cells on said patterned surface and skeletal muscle cells on said gel.

14. The method of claim 13, further comprising c) detecting myo-fiber formation by said skeletal muscle cells.

15. The method of claim 14, wherein said detecting of myo-fiber formation comprises detecting myo-fiber contractions.

16. The method of claim 13, wherein said skeletal muscle cells and/or said motor neurons are exposed to a flow of culture media for a period of time.

17. A microfluidic device comprising a) a membrane, said membrane comprising a top surface and a bottom surface; and b) induced motor neuron cells on said top surface and skeletal muscle cells on said bottom surface.

18. The device of claim 17, wherein said induced motor neuron cells are derived from induced pluripotent stem cells from a human.

19. The device of claim 18, wherein said human is diagnosed with a CNS disorder.

20. The device of claim 19, wherein said CNS disorder is ALS.
